(19) United States
(12) Patent Application Publication
(10) Pub. No.: US 2024/0350981 A1
(43) Pub. Date: Oct. 24, 2024

(54) SELECTIVE AND HYDROGEN-STABLE FACILITATED TRANSPORT MEMBRANES FOR OLEFIN-PARAFFIN SEPARATION

(71) Applicant: BOARD OF REGENTS, THE UNIVERSITY, Austin, TX (US)

(72) Inventors: Benny D. FREEMAN, Austin, TX (US); Joan F. BRENNECKE, Austin, TX (US); Matthew N. DAVENPORT, Austin, TX (US)

(21) Appl. No.: 18/684,307

(22) PCT Filed: Aug. 18, 2022
(86) PCT No.: PCT/US2022/040804

§ 371 Date: Feb. 16, 2024

Related U.S. Application Data
(60) Provisional application No. 63/234,583, filed on Aug. 18, 2021.

(51) Int. Cl. B01D 69/14; B01D 53/22; B01D 69/02; B01D 69/10; B01D 71/02; B01D 71/52; C07C 5/00; C07C 7/144

(52) U.S. Cl. CPC B01D 69/1411; B01D 53/228; B01D 69/02; B01D 69/10; B01D 71/022; B01D 71/5211; C07C 5/00; C07C 7/144; B01D 2257/7022; B01D 2325/34

(57) ABSTRACT

Described are membranes for separating olefins from a mixture that includes olefins and non-olefins. The membrane includes polymers and metal ions associated with the polymers. The metal ions mediate the transport of the olefins through the membrane by selectively and reversibly coupling with the olefins. The olefin/non-olefin selectivity of the membrane remains within at least 80% of its original selectivity after 200 hours of exposure of the membrane to a stream of hydrogen gas, 100 hours of exposure to a stream of acetylene gas, and 100 hours of exposure to a stream of hydrogen sulfide gas. Additional embodiments of the present disclosure pertain to methods of utilizing the membranes of the present disclosure to separate olefins from a mixture that includes olefins and non-olefins.

### CROSS-REFERENCE TO RELATED APPLICATIONS

This application claims priority to U.S. Provisional Patent Application No. 63/234,583, filed on Aug. 18, 2021. The entirety of the aforementioned application is incorporated herein by reference.

### STATEMENT REGARDING FEDERALLY SPONSORED RESEARCH

This invention was made with government support under Grant no. EEC1647722 awarded by the National Science Foundation. The government has certain rights in the invention.

### BACKGROUND

Current systems for separating olefins from non-olefins (e.g., paraffins) have numerous limitations, such as high energy requirements, costly equipment, poor suitability to small-scale applications, and in some cases the need to constantly regenerate and replenish the systems. Various embodiments of the present disclosure address the aforementioned limitations.

### SUMMARY

In some embodiments, the present disclosure pertains to membranes for separating olefins from a mixture that includes olefins and non-olefins. In some embodiments, the membrane includes polymers and metal ions associated with the polymers. In some embodiments, the membrane lacks any ionic liquids. In some embodiments, the metal ions mediate the transport of the olefins through the membrane by selectively and reversibly coupling with the olefins. In some embodiments, the olefin/non-olefin selectivity of the membrane remains within at least 80% of its original selectivity after 200 hours of exposure of the membrane to a stream of hydrogen gas.

In some embodiments, the present disclosure pertains to methods of utilizing the membranes of the present disclosure to separate olefins from a mixture that includes olefins and non-olefins. In some embodiments, the methods of the present disclosure include one or more of the following steps: (1) providing a mixture that contains olefins and non-olefins; (2) associating the mixture with a membrane of the present disclosure, which contains metal ions associated with polymers; (3) utilizing the metal ions to mediate the transport of the olefins through the membrane by selectively and reversibly coupling with the olefins; and (4) reusing the membrane.

### DESCRIPTION OF THE DRAWINGS

FIG. 1 provides a scheme of a method for separating olefins from a mixture.

FIGS. 2A and 2B provide experimental results pertaining to ethylene and ethane pure gas permeability (FIG. 2A) and selectivity (FIG. 2B) as a function of silver bis(trifluoromethanesulfonyl)imide (AgTf₂N) concentration in dense films of cross-linked poly(ethylene glycol) diacrylate (XLPEGDA). Lines are provided to guide the eye. Pure gas ethylene and ethane permeability were measured for several samples of 50 wt % AgTf₂N XLPEGDA80, and the uncertainty was estimated as two standard deviations of the mean (i.e., 95% confidence interval). The relative uncertainty in permeability values for the other AgTf₂N concentrations is estimated based on that of the 50 wt % AgTf₂N sample. The feed pressure was 2 bar, and the permeate was at much lower pressure.

FIG. 3 provides a Robeson plot of the pure gas ethylene-ethane polymeric gas separation membrane upper bound. Values for AgTf₂N solid polymer electrolytes (SPEs) were measured at 2 bar feed pressure and 35° C. The numbers beside these data points represent the concentration of AgTf₂N in the membrane in wt %. Values for other membrane materials from the literature were measured between 20-35° C. and 1-8 bar transmembrane pressure. A dotted line is drawn to indicate an upper bound of polymeric membrane performance according to the well-known tradeoff between membrane permeability and selectivity. A high-performing Ag⁺ SPE facilitated transport membrane from the literature, consisting of 80 wt % AgBF₄ dissolved in the commercial polyether-polyamide copolymer PEBAX 2533, is given for comparison. “Polymeric membranes” denotes polymer membranes without additional fillers, salts, or thermal treatment, “Carbonized membranes” denotes carbon molecular sieve materials where polymeric membranes have been thermally treated, “Ag ionic liquid composites” denotes both polymeric membranes with co-dissolved ionic liquids and silver salts as well as Ag salt-containing ionic liquids supported on a porous and inert support material, and “MOF composites” denotes heterogenous membranes with metal organic framework (MOF) fillers dispersed in a polymer matrix. Data for permeation properties of materials from the literature was sourced from: M. Rungta, et al., AICHE J., vol. 59, no. 9, pp. 3475-3489, 2013; Y.-H. Chu, et al., J. Memb. Sci., 2018, 548, 609-620; M. Rungta, et al., Carbon, vol. 50, no. 4, pp. 1488-1502, 2012; O. Salinas, et al., RSC Adv, vol. 7, no. 6, pp. 3265-3272, 2017; O. Salinas, et al., J. Memb. Sci., vol. 504, pp. 133-140, 2016; L. C. Tomé, et al., J. Mater. Chem. A, vol. 2, no. 16, pp. 5631-5639, 2014; J. E. Bachman, et al., Nature Materials, 2016, 15, 845-849; and I. Pinnau and L. G. Toy, J. Memb. Sci., 2001, 184, 39-48.

FIG. 4 shows the glass transition temperature, T_{g}, of AgTf₂N XLPEGDA80 membranes as a function of AgTf₂N concentration. Glass transition temperatures were reported as the location of the loss modulus peak during a dynamic mechanical analysis (DMA) experiment.

FIGS. 5A and 5B provide pure gas sorption isotherms of ethylene (FIG. 5A) and ethane (FIG. 5B) at 35° C. in both neat XLPEGDA100 and 50 wt % AgTf₂N XLPEGDA80. A dual-mode sorption model based upon chemical equilibrium of Ag⁺-ethylene complexation is presented for ethylene sorption in the AgTf₂N-containing membrane. The rest of the data are fitted by linear regressions through the origin. Error bars are calculated as the average relative difference in ethylene sorption between two different samples of 50 wt % AgTf₂N XLPEGDA80. Error bars are smaller than the markers for ethylene sorption in the neat polymer (FIG. 5A).

FIG. 6 presents equilibrium ethylene and ethane solubility at 35° C. in a 50 wt % AgTf₂N XLPEGDA80 membrane as a function of gas pressure. Error analysis was performed in the same manner as FIG. 5A and FIG. 5B. Ethylene sorption is fitted according to the dual-mode model.

FIGS. 7A and 7B present pure gas permeability and diffusivity data for a 50 wt % AgTf₂N XLPEGDA80 membrane sample. FIG. 7A shows pure gas permeability of ethylene and ethane at 35° C. as a function of transmembrane pressure for a 50 wt % AgTf₂N XLPEGDA80 membrane sample. Uncertainty of the permeability is derived using the procedure described for FIG. 2A. FIG. 7B provides diffusivity calculated from FIG. 7A using the solubilities reported in FIG. 5A and FIG. 5B according to the solution-diffusion model. Uncertainty of the diffusivity is obtained by propagating the uncertainty in the permeability (FIG. 7A) and solubility (FIG. 6) according to the solution diffusion model.

FIG. 8 shows an Ag⁺ carrier stability study of a 50 wt % AgTf₂N XLPEGDA80 membrane. Pure gas C₂H₄ and C₂H₆ permeability, along with the corresponding ideal selectivity are presented as a function of H₂ exposure time. Pure gas C₂H₄ and C₂H₆ permeability measurements were conducted at 35° C. and 2 bar of transmembrane pressure (i.e., the feed pressure was 2 bar). The H₂ permeation experiment (i.e., H₂ exposure experiment) was conducted at 4 bar H₂ feed pressure and 35° C.

FIG. 9 provides a comparison of H₂ stability of ethylene-ethane selectivity between AgTf₂N-containing membranes in this work (i.e., 50 wt % AgTf₂N XLPEGDA80) and state-of-the-art AgBF₄-containing membranes (80 wt % AgBF₄—PEBAX 2533) from the literature (T. C. Merkel, R. Blanc, I. Ciobanu, B. Firat, A. Suwarlim and J. Zeid, Journal of Membrane Science, 2013, 447, 177-189). All measurements were performed at 35° C. and a transmembrane pressure (i.e., feed pressure) of 2 bar. The H₂ permeation experiment was conducted at 2 bar H₂ feed pressure and 35° C. such that the H₂ exposure condition was the same for both membranes.

FIGS. 10A-C provide additional data relating to the characterization of 50 wt % AgTf₂N XLPEGDA80 membranes following exposure to pure H₂ and UV light. FIG. 10A shows a color change of 50 wt % AgTf₂N XLPEGDA80 membranes following 1152 hours of H₂ exposure (black data series in FIG. 10C and same membrane sample as described in FIG. 8). FIG. 10B shows a progressive change in color of 50 wt % AgTf₂N XLPEGDA80 membrane following exposure to 312 nm UV light at an intensity of 3 mW/cm². FIG. 10C shows the x-ray photoelectron spectroscopy (XPS) spectra of the Ag 3d_{5/2} region, which correlates with the oxidation state of Ag⁺ in samples shown in FIGS. 10A and 10B.

FIG. 11 shows an Ag⁺ carrier stability study of a 50 wt % AgTf₂N XLPEGDA80 membrane. Pure gas C₂H₄ and C₂H₆ permeability, along with the corresponding ideal selectivity are presented as a function of C₂H₂ exposure time. Pure gas C₂H₄ and C₂H₆ permeability measurements were conducted at 35° C. and 2 bar of transmembrane pressure (i.e., the feed pressure was 2 bar). The C₂H₂ permeation experiment (i.e., C₂H₂ exposure experiment) was conducted at 2 bar feed pressure with a mixture comprised of 5 vol % C₂H₂ with balance N₂ at 35° C.

FIG. 12 shows an Ag⁺ carrier stability study of a 50 wt % AgTf₂N XLPEGDA80 membrane. Pure gas C₂H₄ and C₂H₆ permeability are presented as a function of H₂S exposure time. Pure gas C₂H₄ and C₂H₆ permeability measurements were conducted at 35° C. and 2 bar of transmembrane pressure (i.e., the feed pressure was 2 bar). The H₂S permeation experiment (i.e., H₂S exposure experiment) was conducted at 2 bar feed pressure with a mixture comprised of 300 ppm H₂S in balance N₂ at 35° C.

FIG. 13 shows an Ag⁺ carrier stability study of a 50 wt % AgTf₂N XLPEGDA80 membrane. The ideal ethylene-ethane selectivity is presented as a function of H₂S exposure time. Pure gas C₂H₄ and C₂H₆ permeability measurements were conducted at 35° C. and 2 bar of feed pressure (FIG. 12) Error bars were obtained by propagating the uncertainty in the C₂H₄ and C₂H₆ pure gas permeabilities. The H₂S permeation experiment (i.e., H₂S exposure experiment) was conducted at 2 bar feed pressure with a mixture comprised of 300 ppm H₂S in balance N₂ at 35° C.

FIGS. 14A-D provide additional data relating to the characterization of 50 wt % AgTf₂N XLPEGDA80 membranes following long-term exposure to H₂S (cf. FIGS. 12 and 13). FIG. 14A shows a 50 wt % AgTf₂N XLPEGDA80 membrane affixed to a brass disc prior to H₂S permeation. FIG. 14B shows the top side of the same membrane (i.e., the surface exposed to the pressurized H₂S-containing mixture) following 264 hours of H₂S exposure and removal from the brass disc. FIG. 14C shows the bottom or reverse side of the same membrane following 264 hours of H₂S exposure. FIG. 14D shows the x-ray photoelectron spectroscopy (XPS) spectra in the Sulfur 2p region of samples shown in FIGS. 14B and 14C, which correlates with the oxidation state of S. In addition, XPS spectra of a control sample similar to that in FIG. 14A, which had not been exposed to H₂S, are provided for comparison. Spectra are displaced vertically for ease of comparison.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Unless stated otherwise, the term “about” means within 5% (e.g., within 2% or 1%) of the particular value modified by the term “about.”

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Olefinic compounds are vital chemical building-blocks for plastics, engineered fluids, surfactants, and plasticizers, and ethylene and propylene are the largest volume organic commodity chemicals. These essential molecules are commercially produced via either steam or catalytic cracking of naphtha, ethane, or propane. Steam cracking is an equilibrium process, and commercial catalytic cracking achieves approximately 30% olefin yield per pass, so purification is required to achieve polymerization grade olefin (97-99% olefin). Other emerging processes, such as oxidative coupling of methane (OCM) and methanol to olefins (MTO), in addition to the more mature Fischer-Tropsch process, also require olefin-paraffin separation. Electrocatalytic reduction of CO₂ to ethylene, does not require olefin-paraffin separation but is not currently practiced industrially due to low energy efficiency and competition with inexpensive natural gas feedstocks. Olefin-paraffin separation is inherently difficult due to the similar molecular sizes and volatility properties of, for example, ethylene and ethane. Consequently, olefin-paraffin separation is usually accomplished via cryogenic distillation at high pressure.

Because of the similar volatilities of corresponding olefin and paraffin gas pairs, the commercial separation process, cryogenic distillation, is energy and capital intensive. Olefin splitters with hundreds of stages operate at sub-zero temperatures and elevated pressure (down to −160° C. and up to 30 bar, respectively), comprising one third of the total energy use in naphtha steam cracking. Overall, ethylene and propylene purification account for 0.3% of global energy use (D. S. Sholl and R. P. Lively, Nature, 2016, 532, 435-437). These separations are essential and ubiquitous in the global chemical industry, and improvements in energy efficiency or process intensification are highly desirable.

Gas separation membranes could offer energy efficiency improvements in comparison to cryogenic distillation because membranes do not require a phase change to affect separation and can be operated at ambient temperature. One immediate application of olefin-paraffin separation membranes could be recovery of the 15% of ethylene that leaves a poly(ethylene) facility as waste gas. Membrane systems could also be used in conjunction with distillation as an additional pathway to improving efficiency and process debottlenecking. Finally, membrane-based olefin-paraffin separation could be an enabling technology for mobile, decentralized natural gas processing plants. It has been estimated that membranes with a mixed-gas ethylene-ethane selectivity ranging from 10-30 could be viable for hybrid distillation processes, with even higher values would be needed to replace cryogenic distillation columns entirely (A. Motelica, O. S. L. Bruinsma, R. Kreiter, M. den Exter and J. F. Vente, Ind. Eng. Chem. Res., 2012, 51, 6977-6986).

However, traditional gas separation materials exhibit poor performance for olefin-paraffin separations. Corresponding olefin-paraffin pairs, such as ethylene and ethane, have similar volatility (as measured by gas critical temperature or normal boiling point) and kinetic diameter, as shown in Table 1. Thus, traditional size-sieving gas separation polymers, such as polyimides, typically exhibit pure gas selectivity values less than 5 for ethylene-ethane separation. Further, Robeson identified a fundamental tradeoff between gas permeability and selectivity across a broad range of polymeric membrane materials, so membranes exhibiting sufficient olefin-paraffin selectivity are hampered by low olefin permeability (M. Rungta, C. Zhang, W. J. Koros and L. Xu, AIChE J., 2013, 59, 3475-3489).

TABLE 1
Thermophysical properties of selected corresponding olefin-
paraffin pairs. (Reid, R. C., Prausnitz, J. M., and Poling, B. E.
The properties of gases and liquids. 1987; J.-R. Li, R. J.
Kuppler and H.-C. Zhou, Chemical Society Reviews, 2009, 38,
1477-1504., K. Tanaka, A. Taguchi, J. Hao, H. Kita and K.
Okamoto, Journal of Membrane Science, 1996, 121, 197-207.)

| | Critical | Normal boiling | Kinetic |
|---|---|---|---|
| Gas | temperature [K] | point [K] | Diameter [Å] |
| Ethylene | 282.4 | 169.3 | 4.16 |
| Ethane | 305.4 | 184.6 | 4.44 |
| Propylene | 364.9 | 225.5 | 4.68 |
| Propane | 369.8 | 231.1 | 5.06 |

Facilitated transport mechanisms have been explored to improve the separation properties of polymeric membranes in several applications, such as incorporation of amine moieties to increase CO₂ transport and metal ion porphyrin complexes to improve O₂ transport. Silver cations (Ag⁺)—as well as other transition metal ions of similar electronic structure—coordinate reversibly with olefin compounds, but not paraffins, to form transition metal-olefin complexes, typically described by the Dewar-Chatt-Duncanson model, i.e., π backbonding. Research has primarily focused on Ag⁺ due to favorable electronegativity for π-complexation and low lattice energy of Ag (I) salts when compared with other transition metal salts. To form an olefin facilitated transport membrane, Ag⁺ is dispersed in a solvating medium, serving as a selective olefin carrier species. Olefin transport is affected by the solvent properties of the medium and the strength of interaction between Ag⁺ and its corresponding anion. Various embodiments of this idea include ion exchange of Ag⁺ in charged membranes, recirculated and regenerated liquid Ag⁺ solutions in gas-liquid contactors, membranes containing ionic liquids (IL) with dissolved Ag (I) salts, and dry solid polymer electrolytes (SPEs) with dissolved Ag (I) salts.

Ag (I) salts are highly soluble in polymers containing ether oxygen moieties due to complexation between the ether oxygen heteroatoms and the Ag⁺ cation, which is often ascribed to differences in their Lewis acidity according to Pearson's hard-soft acid base model. Thus, SPEs formed from polyethers, such as poly(ethylene oxide) (PEO), are a convenient platform for evaluating the performance of different Ag (I) salts for olefin facilitated transport. In a SPE facilitated transport membrane, Ag⁺ ions coordinated to the polymer backbone act as fixed-site carrier species for olefin transport, with olefin molecules reversibly binding to Ag⁺ and transferring between sites throughout the membrane. “Associating”, in this context, means contacting the gas stream or gas streams with the membrane.

Ag⁺ facilitated transport membranes show high olefin-paraffin selectivity. However, the high reactivity of silver ions makes them vulnerable to poisoning by reducing gas species, such as hydrogen (H₂), hydrogen sulfide (H₂S), and acetylene (C₂H₂). Merkel, et al. investigated an SPE consisting of silver tetrafluoroborate (AgBF₄) solvated in a polyether-block-polyamide copolymer (PEBAX 2533). The mixed-gas ethylene/ethane selectivity of an 80 wt % AgBF₄ membrane dropped from approximately 40 to unity after seven days of pure hydrogen permeation at 2 bar. (T. C. Merkel, R. Blanc, I. Ciobanu, B. Firat, A. Suwarlim and J. Zeid, Journal of Membrane Science, 2013, 447, 177-189.)

Industrially, H₂ concentrations between 1-4 wt % are typically found in the olefin-rich stream following dehydrogenation. H₂S is treated prior to cracking in a Residue Desulfurization Unit (RDS), but in some cases the concentration is still approximately 0.2 vol. % in the feed. Likewise, acetylene is hydrogenated back to ethylene, but could still constitute approximately 1 vol. % of the feed and must be removed by distillation. Thus, the propensity for reduction of Ag⁺, often referred to as “carrier instability” in the literature, is one of the primary obstacles to the commercialization of facilitated transport membranes for olefin/paraffin separation.

As such, a need exists for improved methods and systems for separating olefins from a mixture in a selective and continuous manner that remains stable with time. Various embodiments of the present disclosure address this need.

In some embodiments, the present disclosure pertains to membranes for separating olefins from a mixture that includes olefins and non-olefins. In some embodiments, the membrane includes polymers and metal ions associated with the polymers. In some embodiments, the metal ions mediate the transport of the olefins through the membrane by selectively and reversibly coupling with the olefins. In some embodiments, the olefin/non-olefin selectivity of the membrane remains within at least 80% of its original selectivity after 200 hours of exposure of the membrane to a stream of hydrogen gas.

In some embodiments, the present disclosure pertains to methods of utilizing the membranes of the present disclosure to separate olefins from a mixture that includes olefins and non-olefins. In some embodiments illustrated in FIG. 1, the methods of the present disclosure include one or more of the following steps: providing a mixture that contains olefins and non-olefins (step 10); associating the mixture with a membrane of the present disclosure, which contains metal ions associated with polymers (step 12); utilizing the metal ions to mediate the transport of the olefins through the membrane by selectively and reversibly coupling with the olefins (step 14); and reusing the membrane (step 16). In some embodiments, the reusing occurs by continuously associating a mixture with the membrane to produce a continuously flowing permeate stream.

As set forth in more detail herein, the membranes and methods of the present disclosure can have numerous embodiments. In particular, the membranes and methods of the present disclosure can be utilized to separate various olefins from various non-olefins in various mixtures by utilizing various types of metal ions and polymers. Moreover, the metal ions of the present disclosure can mediate the transport of olefins through membranes through various mechanisms.

### Mixtures

In the present disclosure, mixtures generally refer to mixtures that include olefins and non-olefins. The mixtures of the present disclosure can be in various forms. For instance, in some embodiments, the mixtures of the present disclosure can be in gaseous form. In some embodiments, the mixtures of the present disclosure can be in liquid form. In some embodiments, the mixtures of the present disclosure can be in gaseous and liquid forms.

The mixtures of the present disclosure can include various types of non-olefins. For instance, in some embodiments, the non-olefins in the mixture include, without limitation, hydrocarbons, paraffins (i.e., alkanes), carbon dioxide, hydrogen, methane, hydrogen sulfide (H₂S), acetylene, nitrogen, gases thereof, and combinations thereof. In some embodiments, the non-olefins in the mixture include paraffins. In some embodiments, the non-olefins in the mixture include, without limitation, ethane, propane, butane, and combinations thereof.

The mixtures of the present disclosure can also include various types of olefins (i.e., alkenes). For instance, in some embodiments, the olefins in the mixture include dehydrogenated versions of the non-olefins. In some embodiments, the olefins in the mixture include dehydrogenated paraffins. In some embodiments, the olefins in the mixture include, without limitation, ethylene, propylene, butylene, and combinations thereof.

In some embodiments, the olefins in the mixture include ethylene and the non-olefins include ethane. In some embodiments, the olefins in the mixture include propylene and the non-olefins include propane.

The mixtures of the present disclosure can also include various reducing agents. For instance, in some embodiments, the reducing agents include hydrogen gas. In some embodiments, the reducing agents include hydrogen sulfide. In some embodiments, the reducing agents include acetylene.

The mixtures of the present disclosure can include various amounts of reducing agents. For instance, in some embodiments, the mixtures of the present disclosure include trace amounts of reducing agents. In some embodiments, the reducing agents constitute less than 5 wt % of the mixture, less than 1 wt % of the mixture, less than 0.1 wt % of the mixture, less than 0.01 wt % of the mixture, less than 0.001 wt % of the mixture, less than 0.0001 wt % of the mixture, less than 0.00001 wt % of the mixture, or less than 0.000001 wt % of the mixture.

The mixtures of the present disclosure can be derived from various sources. For instance, in some embodiments, the mixtures can be derived from shale gas resources.

### Mixture Dehydrogenation

In some embodiments, the mixtures are provided in untreated form. In some embodiments, the mixtures undergo a dehydrogenation step to result in the formation of olefins from the non-olefins in the mixture. As such, in some embodiments, the methods of the present disclosure also include a step of dehydrogenating the mixture prior to associating the mixture with a membrane.

Dehydrogenation can occur in various manners. For instance, in some embodiments, the dehydrogenation can occur by catalytic dehydrogenation. In some embodiments, dehydrogenation occurs by steam cracking. In some embodiments, steam cracking occurs when steam is added as an inert diluent to a membrane, which in turn shifts the equilibrium towards formation of more olefins.

In some embodiments, the dehydrogenation step generates hydrogen gas. As such, in some embodiments, the methods of the present disclosure also include a step of removing at least some of the generated hydrogen gas from the mixture. In some embodiments, a majority of the generated hydrogen gas is removed from the mixture. In some embodiments, at least 50% of the generated hydrogen gas is removed from the mixture. In some embodiments, at least 75% of the generated hydrogen gas is removed from the mixture. In some embodiments, at least 90% of the generated hydrogen gas is removed from the mixture. In some embodiments, at least 95% of the generated hydrogen gas is removed from the mixture. In some embodiments, at least 99% of the generated hydrogen gas is removed from the mixture. In some embodiments, none of the generated hydrogen gas is removed from the mixture.

Association of Mixtures with Membranes

Various methods can be utilized to associate mixtures with a membrane. For instance, in some embodiments, the associating occurs by contacting the mixture with the membrane. In some embodiments, the associating includes a direct interaction between the mixture and the membrane.

The association of mixtures with a membrane can occur under various conditions. For instance, in some embodiments, the association can occur in the presence of reducing agents, such as hydrogen gas. In some embodiments, the association occurs in the presence of substantial amounts of reducing agents, such as reducing agents that constitute more than 5 wt %, more than 10 wt %, more than 15 wt %, or more than 20 wt % of the mixture. In some embodiments, the association occurs in the presence of trace amounts of reducing agents, such as reducing agents that constitute less than 5 wt %, less than 1 wt %, less than 0.1 wt %, less than 0.01 wt %, less than 0.001 wt %, less than 0.0001 wt %, less than 0.00001 wt % of the mixture, or less than 0.000001 wt % of the mixture.

In some embodiments, the methods of the present disclosure can also include a step of removing various amounts of reducing agents (e.g., hydrogen gas) from a mixture prior to associating the mixture with a membrane. However, in some embodiments, reducing agents are not removed or not completely removed from the mixture prior to associating the mixture with a membrane.

The association of mixtures with a membrane can occur under various temperatures. For instance, in some embodiments, the association can occur at room temperature. In some embodiments, the association can occur above room temperature. In some embodiments, the association can occur at more than about 20° C., more than about 30° C., more than about 40° C., more than about 50° C., more than about 60° C., more than about 70° C., more than about 80° C., more than about 90° C., more than about 100° C., more than about 110° C., more than about 120° C., or more than about 200° C.

In some embodiments, the association can occur below room temperature. For instance, in some embodiments, the association can occur at cryogenic temperatures. In some embodiments, the association can occur at less than about −150° C., less than about −100° C., less than about −85° C., less than about −75° C., less than about −50° C., less than about −40° C., less than about −30° C., less than about −20° C., less about −10° C., less than about 0° C., less than about 5° C., less than about 10° C., less than about 15° C., or less than about 20° C.

In some embodiments, the methods of the present disclosure also include a step of heating or cooling the mixture prior to their association with a membrane. For instance, in some embodiments, the mixtures can be heated in order to reach one or more of the aforementioned temperatures. In some embodiments, the mixtures can be cooled in order to reach one or more of the aforementioned temperatures.

### Olefin Transport

Without being bound by theory, the metal ions of the present disclosure are believed to mediate the transport of olefins through a membrane by selectively and reversibly coupling with the olefin. In some embodiments, the selective and reversible coupling of metal ions with olefins occurs by a reversible interaction between the olefin and the metal ion through π bond complexation. In some embodiments, the metal ions serve as fixed site carriers of the olefins through the membrane.

In some embodiments, the transport of the olefins through the membrane is believed to occur through facilitated transport. In some embodiments, the metal ions serve as fixed site facilitated transport carriers of the olefins through the membrane. In some embodiments, the olefin molecules reversibly bind to different metal ions throughout the membrane during the facilitated transport.

The coupling of metal ions with olefins can have various effects on the transport of olefins through a membrane. For instance, in some embodiments, the coupling increases the solubility of the olefins with respect to the non-olefins, thereby improving the transport of the olefins through the membrane.

### Membranes

The membranes of the present disclosure generally include polymers and metal ions associated with the polymers. The membranes of the present disclosure can be in various forms. For instance, in some embodiments, the membranes of the present disclosure are in the form of non-porous membranes. In some embodiments, the membranes of the present disclosure are in the form of composite membranes.

The membranes of the present disclosure can also include various thicknesses. For instance, in some embodiments, the membranes of the present disclosure include thicknesses that range from about 0.01 μm to about 200 μm. In some embodiments, the membranes of the present disclosure include thicknesses that range from about 0.1 μm to about 200 μm. In some embodiments, the membranes of the present disclosure include thicknesses that range from about 1 μm to about 200 μm. In some embodiments, the membranes of the present disclosure include thicknesses that range from about 10 μm to about 200 μm. In some embodiments, the membranes of the present disclosure include thicknesses that range from about 2 μm to about 175 μm. In some embodiments, the membranes of the present disclosure include thicknesses that range from about 10 μm to about 100 μm. In some embodiments, the membranes of the present disclosure include thicknesses that range from about 50 μm to about 100 μm. In some embodiments, the membranes of the present disclosure include thicknesses of about 60 μm.

In some embodiments, the membranes of the present disclosure are associated with supports. In some embodiments, the supports are porous supports. In some embodiments, the membranes of the present disclosure include non-porous membranes that are associated with porous supports.

The porous supports of the present disclosure can include various porosities. For instance, in some embodiments, the porous supports of the present disclosure have porosity greater than about 0.01%, greater than about 0.02%, greater than about 0.05%, greater than about 0.07%, greater than about 0.1%, greater than about 0.2%, greater than about 0.5%, greater than about 0.7%, greater than about 1.0%, greater than about 1.2%, greater than about 1.5%, greater than about 1.7%, greater than about 2.0%, greater than about 2.2%, greater than about 2.5%, greater than about 2.7%, or greater than about 3.0%. In some embodiments, the porous supports of the present disclosure have porosity less than about 50%.

In some embodiments, the porous supports of the present disclosure have pores with diameters ranging from about 1 nm to about 500 nm. In some embodiments, the porous supports of the present disclosure have pores with diameters ranging from about 1 nm to about 100 nm. In some embodiments, the porous supports of the present disclosure have pores with diameters ranging from about 1 nm to about 50 nm. In some embodiments, the porous supports of the present disclosure have pores with nominal pore size of about 50 nm in diameter.

The membranes of the present disclosure can include various structures. For instance, in some embodiments, the membranes of the present disclosure can be in the form of at least one of a flat disk, a tube, a spiral wound, or a hollow fiber base.

In some embodiments, the membrane is in the form of a homogenous material. In some embodiments, the membrane is rubbery at room temperature and transparent.

In some embodiments, the membranes of the present disclosure consist essentially of polymers, metal ions and counterions. In some embodiments, the membranes of the present disclosure lack ionic liquids.

### Polymers

The membranes of the present disclosure can include numerous types of polymers. For instance, in some embodiments, the polymers include, without limitation, polyamides, polyimides, polyetherimide, polypyrrolones, polyesters, polyethers, poly(vinyl methyl ketone) poly(ether ether ketone), polymethylene oxides, polyethylene oxides, poly(trimethylene oxides), poly(tetramethylene oxides), poly(propylene oxides), polyethylene glycols, poly(ethylene imine), polyalkylene sulfides, sulfone-based polymers, nitrile-based polymers, polymeric organosilicones, fluorinated polymers, polydimethylsiloxane, polydiethylsiloxane, polydi-iso-propylsiloxane, polydiphenylsiloxane, polyethersulfone, polyphenylsulfone, polysulfone, polyacrylonitrile, polyvinylidene fluoride, polyphenylene oxide, polybenzimidazole, polyvinylpyrrolidone, poly(2-oxazoline), poly(ethyl methacrylate), poly(methyl methacrylate), poly(acrylic acid), polyacrylamide, poly(vinyl alcohol), poly(ε-caprolactone), poly(styrene-b-butadiene-b-styrene), chitosan, cellulose acetate, copolymers thereof, and combinations thereof. In some embodiments, the polymers include polyethylene glycols. In some embodiments, the polymers include poly(ethylene glycol) diacrylate.

The polymers of the present disclosure can have various sizes. For instance, in some embodiments, the polymers of the present disclosure have molecular weights ranging from about 400 Da to about 2,000 Da when in un-cross-linked form. In some embodiments, the polymers of the present disclosure include molecular weights ranging from about 400 Da to about 1,000 Da when in un-cross-linked form. In some embodiments, the polymers of the present disclosure include molecular weights ranging from about 400 Da to about 700 Da when in un-cross-linked form. In some embodiments, the polymers of the present disclosure include molecular weights of about 700 Da when in un-cross-linked form.

In some embodiments, the polymers of the present disclosure have molecular weights of less than about 10,000,000 Da when in un-cross-linked form. In some embodiments, the polymers of the present disclosure have molecular weights of less than about 5,000,000 Da when in un-cross-linked form. In some embodiments, the polymers of the present disclosure have molecular weights of less than about 1,000,000 Da when in un-cross-linked form. In some embodiments, the polymers of the present disclosure have molecular weights of less than about 900,000 Da when in un-cross-linked form. In some embodiments, the polymers of the present disclosure have molecular weights of less than about 800,000 Da when in un-cross-linked form. In some embodiments, the polymers of the present disclosure have molecular weights of less than about 700,000 Da when in un-cross-linked form. In some embodiments, the polymers of the present disclosure have molecular weights of less than about 500,000 Da when in un-cross-linked form. In some embodiments, the polymers of the present disclosure have molecular weights of less than about 100,000 Da when in un-cross-linked form. In some embodiments, the polymers of the present disclosure have molecular weights of less than about 50,000 Da when in un-cross-linked form. In some embodiments, the polymers of the present disclosure have molecular weights of less than about 25,000 Da when in un-cross-linked form. In some embodiments, the polymers of the present disclosure have molecular weights of less than about 10,000 Da when in un-cross-linked form. In some embodiments, the polymers of the present disclosure have molecular weights of less than about 2,000 Da when in un-cross-linked form. In some embodiments, the polymers of the present disclosure have molecular weights of less than about 1,000 Da when in un-cross-linked form. In some embodiments, the polymers of the present disclosure have molecular weights of less than about 500 Da when in un-cross-linked form.

The polymers of the present disclosure can be in various forms. For instance, in some embodiments, the polymers of the present disclosure are in cross-linked form. In some embodiments, the polymers of the present disclosure are in the form of a three-dimensional cross-linked network. In some embodiments, the polymers of the present disclosure are in the form of a polymer matrix. In some embodiments, the polymers of the present disclosure include cross-linked polyethylene glycols. In some embodiments, the polymers of the present disclosure are in the form of linear molecules that are not cross-linked.

### Metal Ions

The membranes of the present disclosure can include various types of metal ions. For instance, in some embodiments, the metal ions include, without limitation, transition metal ions, silver ions, copper ions, gold ions, nickel ions, iron ions, manganese ions, zinc ions, and combinations thereof.

In some embodiments, the metal ions of the present disclosure can be associated with counterions. In some embodiments, the metal ions and the counterions have a net neutral charge. In some embodiments, the metal ions and the counterions are in the form of ionic aggregates. In some embodiments, the metal ions and counterions have a net neutral charge.

In some embodiments, the metal ions include metal cations. In some embodiments, the metal cations are associated with anions. In some embodiments, the anions include weakly-coordinating anions. In some embodiments, the weakly-coordinating anions exhibit charge delocalization such that interaction with the cation is low and not competitive with olefin complexation or coordination with the polymer chain. In some embodiments, the weakly-coordinating anions are also known as non-coordinating anions. In some embodiments, the weakly coordinating anions include, without limitation, bis(trifluoromethanesulfonyl)imide (Tf₂N⁻), trifluoromethanesulfonate (TfO⁻), bis(fluorosulfonyl)imide (FSI⁻), hexafluorophosphate (PF₆⁻), and combinations thereof.

In some embodiments, the metal cations and the anions are in the form of ionic aggregates. In some embodiments, the metal cations and anions have a net neutral charge.

In some embodiments, the metal ions of the present disclosure are in the form of metal salts. In some embodiments, the metal salts include silver cations associated with anions. In some embodiments, the metal salts of the present disclosure include, without limitation, AgF, AgBr, AgI, Ag₂CO₃, AgHCO₃, AgNO₂, AgNO₃, Ag₂SO₄, AgClO₄, AgCN, AgSCN, AgOCN, AgAsF₆, AgSbF₆, AgPF₆, AgP(CF₃CF₂)₃F₃, AgBF₄, AgB(CN)₄, AgBF₃(CF₂CF₃), AgB(C₆F₅)₄, AgTfO (AgCF₃SO₃), AgNfO (AgC₄F₉SO₃), AgTf₂N (Ag(CF₃SO₂)₂N), Ag(CF₃)₂N, AgCF₃CO₂, AgN(CN)₂, AgFSI (Ag(FSO₂)₂N), AgC(CN)₃ and combinations thereof. In some embodiments, the metal salts include AgTfO (AgCF₃SO₃), AgNfO (AgC₄F₉SO₃), AgTf₂N (Ag(CF₃SO₂)₂N), Ag(CF₃)₂N, AgCF₃CO₂, AgN(CN)₂, AgN(CF₃CF₂SO₂)₂, AgFSI (Ag(FSO₂)₂N), and AgC(CN)₃. In some embodiments, the metal salts include AgTfO. In some embodiments, the metal salts include AgTf₂N.

The metal ions of the present disclosure can be arranged in the membranes of the present disclosure in various manners. For instance, in some embodiments, the metal ions of the present disclosure are dispersed throughout the polymers of the present disclosure. In some embodiments, the metal ions of the present disclosure are dissolved within the polymers of the present disclosure. In further embodiments, the metal ions are dissolved in the polymers of the membrane.

In some embodiments, the metal ions of the present disclosure are substantially immobile when associated with the polymers of the present disclosure. In some embodiments, the metal ions of the present disclosure are able to diffuse throughout a polymer matrix. In some embodiments, the metal ions of the present disclosure are evenly distributed throughout the polymers. For instance, in some embodiments, the metal ions of the present disclosure are evenly distributed with distances of at least about 9.3 Å between the metal ions. In some embodiments, the metal ions of the present disclosure are evenly distributed with distances of less than about 9.3 Å between the metal ions. In some embodiments, the metal ions of the present disclosure are evenly distributed with distances of less than about 8.5 Å between the metal ions. In some embodiments, the metal ions of the present disclosure are evenly distributed with distances of less than about 8.1 Å between the metal ions. In some embodiments, the metal ions of the present disclosure are evenly distributed with distances of less than about 7.5 Å between the metal ions. In some embodiments, the metal ions of the present disclosure are evenly distributed with distances of less than about 5 Å between the metal ions. In some embodiments, the metal ions of the present disclosure are evenly distributed with distances of less than about 2.5 Å between the metal ions.

In some embodiments, the metal ions of the present disclosure are in solid form. In some embodiments, the metal ions of the present disclosure are not dissolved in ionic liquids. In some embodiments, the metal ions of the present disclosure are dissolved in the polymers of the present disclosure.

The membranes of the present disclosure can have various concentrations of metal ions. For instance, in some embodiments, the metal ions of the present disclosure have concentrations of at least 5 wt % relative to the polymers of the present disclosure. In some embodiments, the metal ions of the present disclosure have concentrations of at least 10 wt % relative to the polymers of the present disclosure. In some embodiments, the metal ions of the present disclosure have concentrations of at least 15 wt % relative to the polymers of the present disclosure. In some embodiments, the metal ions of the present disclosure have concentrations of at least 20 wt % relative to the polymers of the present disclosure. In some embodiments, the metal ions of the present disclosure have concentrations of at least 25 wt % relative to the polymers of the present disclosure. In some embodiments, the metal ions of the present disclosure have concentrations of at least 30 wt % relative to the polymers of the present disclosure. In some embodiments, the metal ions of the present disclosure have concentrations of at least 35 wt % relative to the polymers of the present disclosure. In some embodiments, the metal ions of the present disclosure have concentrations of at least 40 wt % relative to the polymers of the present disclosure. In some embodiments, the metal ions of the present disclosure have concentrations of at least 45 wt % relative to the polymers of the present disclosure. In some embodiments, the metal ions of the present disclosure have concentrations of at least 50 wt % relative to the polymers of the present disclosure. In some embodiments, the metal ions of the present disclosure have concentrations of at least 55 wt % relative to the polymers of the present disclosure. In some embodiments, the metal ions of the present disclosure have concentrations of at least 60 wt % relative to the polymers of the present disclosure. In some embodiments, the metal ions of the present disclosure have concentrations of at least 65 wt % relative to the polymers of the present disclosure. In some embodiments, the metal ions of the present disclosure have concentrations of at least 70 wt % relative to the polymers of the present disclosure. In some embodiments, the metal ions of the present disclosure have concentrations of at least 75 wt % relative to the polymers of the present disclosure.

### Selectivity

The membranes of the present disclosure can have various olefin/non-olefin selectivities. For instance, in some embodiments, the membranes of the present disclosure have a pure gas olefin/non-olefin selectivity of at least about 5 at 35° C. and a feed pressure of 2 bar. In some embodiments, the membranes of the present disclosure have a pure gas olefin/non-olefin selectivity of at least about 5.5 at 35° C. and a feed pressure of 2 bar. In some embodiments, the membranes of the present disclosure have a pure gas olefin/non-olefin selectivity of at least about 6 at 35° C. and a feed pressure of 2 bar. In some embodiments, the membranes of the present disclosure have a pure gas olefin/non-olefin selectivity of at least about 6.5 at 35° C. and a feed pressure of 2 bar. In some embodiments, the membranes of the present disclosure have a pure gas olefin/non-olefin selectivity of at least about 7 at 35° C. and a feed pressure of 2 bar. In some embodiments, the membranes of the present disclosure have a pure gas olefin/non-olefin selectivity of at least about 10 at 35° C. and a feed pressure of 2 bar. In some embodiments, the membranes of the present disclosure have a pure gas olefin/non-olefin selectivity of at least about 15 at 35° C. and a feed pressure of 2 bar. In some embodiments, the membranes of the present disclosure have a pure gas olefin/non-olefin selectivity of at least about 20 at 35° C. and a feed pressure of 2 bar. In some embodiments, the membranes of the present disclosure have a pure gas olefin/non-olefin selectivity of at least about 25 at 35° C. and a feed pressure of 2 bar.

### Hydrogen Stability

The membranes of the present disclosure have high hydrogen stability. For instance, in some embodiments, the olefin/non-olefin selectivity of a membrane of the present disclosure remains within at least 80% of its original selectivity after 200 hours of exposure to a stream of hydrogen gas. In some embodiments, the olefin/non-olefin selectivity of a membrane of the present disclosure remains within at least 80% of its original selectivity after 400 hours of exposure to a stream of hydrogen gas. In some embodiments, the olefin/non-olefin selectivity of a membrane of the present disclosure remains within at least 80% of its original selectivity after 600 hours of exposure to a stream of hydrogen gas. In some embodiments, the olefin/non-olefin selectivity of a membrane of the present disclosure remains within at least 80% of its original selectivity after 800 hours of exposure to a stream of hydrogen gas. In some embodiments, the olefin/non-olefin selectivity of a membrane of the present disclosure remains within at least 80% of its original selectivity after 900 hours of exposure to a stream of hydrogen gas. In some embodiments, the olefin/non-olefin selectivity of a membrane of the present disclosure remains within at least 80% of its original selectivity after 1,200 hours of exposure to a stream of hydrogen gas. In some embodiments, the olefin/non-olefin selectivity of a membrane of the present disclosure remains within at least 80% of its original selectivity after 1,400 hours of exposure to a stream of hydrogen gas.

In some embodiments, the olefin/non-olefin selectivity of a membrane of the present disclosure remains within at least 85% of its original selectivity after 200 hours of exposure to a stream of hydrogen gas. In some embodiments, the olefin/non-olefin selectivity of a membrane of the present disclosure remains within at least 85% of its original selectivity after 400 hours of exposure to a stream of hydrogen gas. In some embodiments, the olefin/non-olefin selectivity of a membrane of the present disclosure remains within at least 85% of its original selectivity after 600 hours of exposure to a stream of hydrogen gas. In some embodiments, the olefin/non-olefin selectivity of a membrane of the present disclosure remains within at least 85% of its original selectivity after 800 hours of exposure to a stream of hydrogen gas. In some embodiments, the olefin/non-olefin selectivity of a membrane of the present disclosure remains within at least 85% of its original selectivity after 900 hours of exposure to a stream of hydrogen gas. In some embodiments, the olefin/non-olefin selectivity of a membrane of the present disclosure remains within at least 85% of its original selectivity after 1,200 hours of exposure to a stream of hydrogen gas. In some embodiments, the olefin/non-olefin selectivity of a membrane of the present disclosure remains within at least 85% of its original selectivity after 1,400 hours of exposure to a stream of hydrogen gas.

In some embodiments, the olefin/non-olefin selectivity of a membrane of the present disclosure remains within at least 90% of its original selectivity after 200 hours of exposure to a stream of hydrogen gas. In some embodiments, the olefin/non-olefin selectivity of a membrane of the present disclosure remains within at least 90% of its original selectivity after 400 hours of exposure to a stream of hydrogen gas. In some embodiments, the olefin/non-olefin selectivity of a membrane of the present disclosure remains within at least 90% of its original selectivity after 600 hours of exposure to a stream of hydrogen gas. In some embodiments, the olefin/non-olefin selectivity of a membrane of the present disclosure remains within at least 90% of its original selectivity after 800 hours of exposure to a stream of hydrogen gas. In some embodiments, the olefin/non-olefin selectivity of a membrane of the present disclosure remains within at least 90% of its original selectivity after 900 hours of exposure to a stream of hydrogen gas. In some embodiments, the olefin/non-olefin selectivity of a membrane of the present disclosure remains within at least 90% of its original selectivity after 1,200 hours of exposure to a stream of hydrogen gas. In some embodiments, the olefin/non-olefin selectivity of a membrane of the present disclosure remains within at least 90% of its original selectivity after 1,400 hours of exposure to a stream of hydrogen gas.

In some embodiments, the olefin/non-olefin selectivity of a membrane of the present disclosure remains within at least 95% of its original selectivity after 200 hours of exposure to a stream of hydrogen gas. In some embodiments, the olefin/non-olefin selectivity of a membrane of the present disclosure remains within at least 95% of its original selectivity after 400 hours of exposure to a stream of hydrogen gas. In some embodiments, the olefin/non-olefin selectivity of a membrane of the present disclosure remains within at least 95% of its original selectivity after 600 hours of exposure to a stream of hydrogen gas. In some embodiments, the olefin/non-olefin selectivity of a membrane of the present disclosure remains within at least 95% of its original selectivity after 800 hours of exposure to a stream of hydrogen gas. In some embodiments, the olefin/non-olefin selectivity of a membrane of the present disclosure remains within at least 95% of its original selectivity after 900 hours of exposure to a stream of hydrogen gas. In some embodiments, the olefin/non-olefin selectivity of a membrane of the present disclosure remains within at least 95% of its original selectivity after 1,200 hours of exposure to a stream of hydrogen gas. In some embodiments, the olefin/non-olefin selectivity of a membrane of the present disclosure remains within at least 95% of its original selectivity after 1,400 hours of exposure to a stream of hydrogen gas.

In some embodiments, the stream of hydrogen gas represents a constant stream of hydrogen gas. In some embodiments, the stream of hydrogen gas represents a stream of pure hydrogen gas. In some embodiments, the stream of hydrogen gas includes a steady stream of hydrogen gas of at least 2 bar. In some embodiments, the stream of hydrogen gas includes a steady stream of hydrogen gas of at least 2 bar at 35° C. In some embodiments, the stream of hydrogen gas includes a steady stream of hydrogen gas of at least 4 bar at 35° C.

### Acetylene and Hydrogen Sulfide Stability

The membranes of the present disclosure have high tolerance towards acetylene and hydrogen sulfide. For instance, in some embodiments, the olefin/non-olefin selectivity of a membrane of the present disclosure remains within at least 80% of its original selectivity after 100 hours of exposure to a stream of acetylene and/or hydrogen sulfide. In some embodiments, the olefin/non-olefin selectivity of a membrane of the present disclosure remains within at least 80% of its original selectivity after 200 hours of exposure to a stream of acetylene and/or hydrogen sulfide. In some embodiments, the olefin/non-olefin selectivity of a membrane of the present disclosure remains within at least 80% of its original selectivity after 500 hours of exposure to a stream of acetylene and/or hydrogen sulfide.

In some embodiments, the olefin/non-olefin selectivity of a membrane of the present disclosure remains within at least 85% of its original selectivity after 100 hours of exposure to a stream of acetylene and/or hydrogen sulfide. In some embodiments, the olefin/non-olefin selectivity of a membrane of the present disclosure remains within at least 85% of its original selectivity after 200 hours of exposure to a stream of acetylene and/or hydrogen sulfide. In some embodiments, the olefin/non-olefin selectivity of a membrane of the present disclosure remains within at least 85% of its original selectivity after 500 hours of exposure to a stream of acetylene and/or hydrogen sulfide.

In some embodiments, the olefin/non-olefin selectivity of a membrane of the present disclosure remains within at least 90% of its original selectivity after 100 hours of exposure to a stream of acetylene and/or hydrogen sulfide. In some embodiments, the olefin/non-olefin selectivity of a membrane of the present disclosure remains within at least 90% of its original selectivity after 200 hours of exposure to a stream of acetylene and/or hydrogen sulfide. In some embodiments, the olefin/non-olefin selectivity of a membrane of the present disclosure remains within at least 90% of its original selectivity after 500 hours of exposure to a stream of acetylene and/or hydrogen sulfide.

In some embodiments, the olefin/non-olefin selectivity of a membrane of the present disclosure remains within at least 95% of its original selectivity after 100 hours of exposure to a stream of acetylene and/or hydrogen sulfide. In some embodiments, the olefin/non-olefin selectivity of a membrane of the present disclosure remains within at least 95% of its original selectivity after 200 hours of exposure to a stream of acetylene and/or hydrogen sulfide. In some embodiments, the olefin/non-olefin selectivity of a membrane of the present disclosure remains within at least 95% of its original selectivity after 500 hours of exposure to a stream of acetylene and/or hydrogen sulfide.

In some embodiments, the stream of acetylene and/or hydrogen sulfide represents a constant stream of acetylene and/or hydrogen sulfide. In some embodiments, the stream of acetylene and/or hydrogen sulfide represents a stream of pure acetylene and/or hydrogen sulfide. In some embodiments, the stream of acetylene and/or hydrogen sulfide includes a steady stream of acetylene and/or hydrogen sulfide of at least 2 bar. In some embodiments, the stream of acetylene and/or hydrogen sulfide includes a steady stream of acetylene and/or hydrogen sulfide of at least 2 bar at 35° C. In some embodiments, the stream of acetylene and/or hydrogen sulfide includes a steady stream of acetylene and/or hydrogen sulfide of at least 4 bar at 35° C.

In some embodiments, the stream of hydrogen sulfide can be a gas mixture that contains about 2 vol %. In other embodiments, the stream of hydrogen sulfide can be a gas mixture that contains from about 0.5 ppm to about 20000 ppm of hydrogen sulfide. In other embodiments, the stream of hydrogen sulfide can be a gas mixture that contains from about 10 ppm to about 10000 ppm of hydrogen sulfide. In other embodiments, the stream of hydrogen sulfide can be a gas mixture that contains from about 100 ppm to about 1000 ppm of hydrogen sulfide. In other embodiments, the stream of hydrogen sulfide can be a gas mixture that contains from about 0.5 ppm to about 1000 ppm of hydrogen sulfide. In other embodiments, the stream of hydrogen sulfide can be a gas mixture that contains from about 1000 ppm to about 20000 ppm of hydrogen sulfide. In other embodiments, the stream of hydrogen sulfide can be a gas mixture that contains from about 0.5 to about 2.5 ppm of hydrogen sulfide. In other embodiments, the stream of hydrogen sulfide can be a gas mixture that contains from about 50 to about 2000 ppm of hydrogen sulfide. In other embodiments, the stream of hydrogen sulfide can be a gas mixture that contains about 0.5, 1.0, 1.5, or 2 vol % of hydrogen sulfide.

In some embodiments, the stream of acetylene can be a gas mixture that contains from about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8., 1.9, or 2.0 vol. % acetylene, where any of the stated values can form an upper or lower endpoint of a range.

### Applications

The methods and systems of the present disclosure can provide numerous advantages. For instance, the systems and methods of the present disclosure provide the ability to maintain olefin-paraffin selectivity following extensive exposure to hydrogen gas. In particular, previously published studies of Ag⁺ containing facilitated transport membranes noted a rapid decline in and near total loss of olefin-paraffin selectivity after one week of H₂ permeation due to reduction of chemically active Ag⁺ ions to silver metal (i.e., Ag⁰), which does not have a specific affinity for olefins. On the other hand, the systems and methods of the present disclosure have not shown any such losses of olefin-paraffin selectivity after over sixty days of total H₂ permeation.

The methods and systems of the present disclosure provide additional advantages. For instance, in some embodiments, the methods and systems of the present disclosure provide a selective and modular membrane technology that can reduce both energy and capital costs significantly relative to cryogenic distillation. In some embodiments, the methods and systems of the present disclosure provide for a relatively inexpensive membrane separation system that could be rapidly implemented to capture olefins from vent streams and improve process efficiency and yield. Additionally, in some embodiments, the methods and systems of the present disclosure provide for a scalable method of separating olefins from non-olefins.

### EXAMPLES

Reference will now be made to more specific embodiments of the present disclosure and experimental results that provide support for such embodiments. However, Applicants note that the disclosure below is for illustrative purposes only and is not intended to limit the scope of the claimed subject matter in any way.

### Example 1. Hydrogen-Stable Ag⁺ Solid Polymer Electrolyte Membranes for Olefin-Paraffin Separations

H₂-stable solid polymer electrolytes of crosslinked poly(ethylene glycol) diacrylate and up to 70 wt % AgTf₂N salt were synthesized through a facile and scalable UV-crosslinking process. Following over 1000 hours of H₂ permeation at 2 bar, the ethylene-ethane selectivity of the membrane remained unchanged, and X-ray photoelectron spectroscopy showed no change in the oxidation state of the dissolved Ag⁺ olefin carriers. At the highest AgTf₂N concentration, pure-gas ethylene-ethane selectivity and ethylene permeability were 20.8 and 4.0 Barrer, respectively, yielding performance surpassing the upper bound for polymeric materials. Pure-gas solubility measurements show high ethylene-ethane solubility as well as dual-mode ethylene sorption despite the rubbery nature of the matrix. Diffusion coefficients were calculated according to the solution-diffusion model.

The pure gas propylene-propane selectivity of supported ionic liquid membranes (SILMs) containing immobilized ionic liquids with silver bis(trifluoromethanesulfonyl)imide, or silver bistriflimide (AgTf₂N), is stable to hydrogen exposure (C. M. Sanchez, T. Song, J. F. Brennecke, and B. D. Freeman, Ind. Eng. Chem. Res., 2020, 59, 5362-5370.) Studies on electrodeposition of silver films from ionic liquids have found that Ag⁺ can be resistant to reduction when dissolved in ionic liquids. For example, cyclic voltammetry experiments on 1-Ethyl-3-methylimidazolium bistriflimide ([EMIM⁺][Tf₂N⁻]) solutions with dissolved AgTf₂N shows a large electrochemical overpotential for reduction from Ag⁺ to Ag⁰, corroborating qualitative evidence of a steric effect inhibiting nanoparticle nucleation. (Liu, Tomin, Electrodeposition of metallic thin films from ionic liquid solutions for electronic applications, 2014, Dissertation, University of Bordeaux, France.)

Tf₂N⁻ is a relatively weakly-coordinating anion, i.e., it exhibits charge delocalization such that interaction strength with the cation is low. Forthcoming research indicates that ionic aggregates of Ag⁺ and Tf₂N⁻ in the ionic liquid solution leads to this steric effect, with their presence correlated with H₂ stability. Incorporating this Ag⁺ stabilization effect into industrially viable polymeric membranes is challenging. SILMs rely on capillary forces to immobilize the ionic liquid solution in the membrane pores, practically limiting the applicable transmembrane pressure to below 2 bar. In addition, polymeric membranes with dissolved ionic liquids can exhibit phase separation and reduced mechanical strength, with the ionic liquid acting as a plasticizing agent. However, the same AgTf₂N salt from the H₂-stable SILMs can be directly dissolved into polyethers at high weight loadings. To investigate the hydrogen stability of these preparations, solid polymer electrolytes (SPEs) of AgTf₂N in crosslinked poly(ethylene glycol) diacrylate (PEGDA) were synthesized and characterized for facilitated transport of ethylene, pure gas ethylene-ethane selectivity, and long-term hydrogen stability.

### Example 1.1. Materials

AgTf₂N was synthesized via an ion exchange reaction between Ag₂O (Strem Chemicals, 99%) and bistriflimic acid (H-Tf₂N), which was in turn obtained by reaction between lithium bistriflimide (LiTf₂N, Sigma-Aldrich, 99.95%) and sulfuric acid (Aldon Corp., 18 M). LiTf₂N was dissolved in concentrated sulfuric acid to form a 30 wt % solution, stirred at 90° C. for three hours, and then vacuum distilled at 90° C. 103.2 g of the product was dissolved in 100 mL of deionized water, and 85.7 g Ag₂O was subsequently added (1:1 molar ratio between H-Tf₂N and Ag₂O) before heating the mixture to 60° C. and stirring for two hours. Water was removed via a rotary evaporator, and the product was dried at 50° C. under high vacuum. 141.2 g of AgTf₂N was obtained as a white solid (99% yield) and found to be free of LiTf₂N impurities using an Agilent 710-ES Inductively Coupled Plasma Optical Emission Spectrometer (ICP-OES) (Agilent Technologies, Santa Clara, CA). Acetonitrile (99.8%), 1-hydroxycyclohexyl phenyl ketone (HCPK) (99%), and poly(ethylene glycol diacrylate) of molecular weight 700 (PEGDA₇₀₀) were purchased from Sigma-Aldrich and used without further purification. The purity of HCPK was confirmed to be at least >99.9% via gas chromatography followed by mass spectrometry (GC-MS). The purity of PEGDA₇₀₀ was confirmed to be greater than 99% via quantitative analysis of the nuclear magnetic resonance (NMR) spectra in deuterated chloroform (CDCl₃) recorded on a 400 MHz Agilent MR spectrometer. ICP-OES, GC-MS, and NMR spectra are presented in the supporting information. Ultra-high purity (99.9%) ethylene and hydrogen were purchased from Airgas, and ultra-high purity ethane was purchased from Matheson. All gases were used without further purification.

### Example 1.2. Membrane Synthesis

Crosslinked PEGDA (XLPEGDA) membranes containing a specified quantity of dissolved AgTf₂N salt were synthesized via a UV crosslinking procedure. Prior to polymerization, a solution containing a fixed quantity of AgTf₂N, PEGDA₇₀₀, and HCPK initiator was stirred for at least one hour. For samples containing higher concentrations of salt, a fixed quantity of acetonitrile was added to the pre-polymerization solution in order to increase salt solubility. Details for each sample and an explanation of the terminology used is provided in Table 2.

TABLE 2
Description of AgTf₂N XLPEGDA membranes synthesized for gas permeation
studies. AgTf₂N concentrations correspond to final compositions of dry
membranes and EO signifies ethylene oxide repeat units in the polymer.

| | | AgTf₂N | Mass ratio of |
|---|---|---|---|
| | AgTf₂N | concentration | acetonitrile relative |
| | concentration | [mol Ag⁺:mol | to PEGDA₇₀₀ |
| Membrane | [% wt.] | EO] | [solvent:polymer] |
| XLPEGDA100 | 0 | — | 0:100 |
| 19.5 wt % AgTf₂N XLPEGDA100 | 19.5 | 1:29.8 | 0:100 |
| 33% wt % AgTf₂N XLPEGDA100 | 33 | 1:14.6 | 0:100 |
| 50% wt % AgTf₂N XLPEGDA80 | 50 | 1:7.2 | 20:80 |
| 60% wt % AgTf₂N XLPEGDA80 | 60 | 1:4.8 | 20:80 |
| 70% wt % AgTf₂N XLPEGDA66 | 70 | 1:3.6 | 33:66 |

A quantity (e.g., 0.2 mL) of pre-polymerization solution was sandwiched between two UV-transparent quartz plates, and crosslinking was performed in a Fischer Scientific FB UVXL-1000 crosslinking oven with 90 s of exposure to UV light with a wavelength of 312 nm at an intensity of 3 mW/cm². Calibrated stainless steel spacers were used to control the thickness of the resulting film and ensure uniformity. Upon exposure to UV light, a free radical polymerization is initiated, reacting the acrylate groups on either end of the di-functionalized PEGDA₇₀₀ monomer to form a three-dimensional crosslinked network.

Extraction with a solvent to remove any non-polymerized monomer or other species not bound to the network (sol), was not performed due to the potential of also removing dissolved AgTf₂N. Following polymerization, the membranes were peeled from the casting plates and dried under vacuum on the order of 1 Pa overnight at ambient temperature, with precautions taken to limit exposure to light. The resulting films were transparent, mechanically robust, and flexible with no discoloration that might indicate reduction of Ag⁺ due to UV exposure during crosslinking.

### Example 1.3. Pure Gas Permeation Studies

Films of uniform thickness were epoxied to brass support disks with a filter paper backing. Film thickness was measured using digital calipers (Mitutoyo, ±1 μm resolution) and ranged from 40 to 200 μm. Membranes with a thickness of 200 μm were easier to handle and developed defects less frequently than thinner samples but could also exhibit gas flux below the measurement threshold of the apparatus at higher AgTf₂N loadings. Membranes were loaded into a high-pressure Millipore filter holder, serving as the permeation cell as part of a constant-volume, variable-pressure permeation system. Samples were degassed overnight at 35° C. to remove any sorbed gas from the sample. The pure-gas permeabilities of H₂, ethylene, and ethane at 35° C. were measured at several transmembrane pressure points ranging from 2 to 8 bar. The rise in downstream pressure due to gas flux was measured with an MKS Baratron with a 10 Torr range, and the upstream pressure was measured with a Honeywell STJE transducer with a 1000 psig [68 bar] range. Gas permeability is reported in units of Barrer, where 1 Barrer=10⁻¹⁰ cm³ (STP) cm cm⁻² s⁻¹ cmHg⁻¹.

### Example 1.4. Mixed Gas Permeation Studies

Samples were prepared with the same method as in the pure gas permeation studies. Membranes were loaded into a high-pressure Millipore filter holder, serving as the permeation cell as part of a constant-pressure, variable-volume permeation system, with a nitrogen sweep and 1% stage cut on both the permeate and retentate. A mixture containing 65/35% vol. ethylene/ethane was obtained using calibrated mass flow controllers and contacted with the upstream face. The mixed gas permeabilities of ethylene and ethane were measured at 3.5 and 5 bar of transmembrane pressure at a temperature of 35° C. A sample of the permeate was sent to a gas chromatograph (GC) in order to determine the composition. The GC column was a ShinCarbon ST with the following dimensions: length: 2 m, outer diameter: ⅛″, inner diameter: 2 mm. Gas permeation was taken to be at steady state once the GC peak area was constant for 30 min.

### Example 1.5. Gas Sorption

A gravimetric method was used to determine ethylene and ethane solubility in the polymer electrolyte membranes. A magnetic suspension balance (MSB) manufactured by Rubotherm GmbH was used to monitor the weight change at a fixed temperature and pressure. Approximately 0.5 g of sample was added to the sample bucket and degassed to roughly 10⁻⁵ bar. After water and any other volatile impurities were evaporated, and the measured weight was constant for at least 2 hours, the chamber was pressurized in incremental steps with either ethylene or ethane to a maximum pressure of 14 bar. Equilibrium between the challenge gas and the polymer electrolyte sample was considered to be achieved once the measured weight remained constant for at least 2 hours. All values were corrected for buoyancy effects.

### Example 1.6. Density Measurements

Density of the samples was measured at ambient temperature via expansion of low-pressure helium into a calibrated chamber containing the polymer sample using a Micromeritics Accupync II 1345 gas pycnometer.

### Example 1.7. Glass Transition Temperature

The glass transition temperature (T_{g}) of the solid polymer electrolyte membranes was measured via dynamic mechanical analysis (DMA) using a DMA Q-800 manufactured by TA instruments. Rectangular coupons approximately 2 cm long and 1 cm wide with a thickness of approximately 200 μm were clamped into the tensile-mode testing apparatus. Samples were cooled to −140° C. and held isothermally for 5 min before ramping to 0° C. at a rate of 1.0° C./min while oscillating at a constant amplitude of 15 μm with a frequency of 1 Hz. The loss modulus was measured over the temperature range, with the location of the peak value taken as the T_{g}. The accuracy of the technique was verified by comparing the T_{g} of the neat polymer sample with a value obtained via a similar procedure from the scientific literature (H. Lin, T. Kai, B. D. Freeman, S. Kalakkunnath, and D. S. Kalika, Macromolecules, 2005, 38, 8381-8393).

### Example 1.8. X-Ray Photoelectron Spectroscopy (XPS)

XPS analyses were performed using a Kratos Axis Ultra DLD XPS, equipped with an Al Kα monochromatic X-ray source with a power set at 120 W. The photoelectrons were collected with an emission angle (EA) of 90° and from a sample area of 300 μm×700 μm. For high-resolution spectra, the measurements were performed in constant-analyzer-energy (CAE) mode with a pass energy of 20 eV and a step size of 0.1 eV (full-width-at-half-maximum of the peak for Ag 3d_{5/2} is 0.77 eV). Survey spectra were collected using a pass energy of 160 eV and a step size of 1 eV. The residual pressure in the analytical chamber was ˜5×10⁻⁹ Torr. The instrument was calibrated according to ISO 15472:2001 with an accuracy of +0.1 eV. The high-resolution spectra were processed using CasaXPS (v2.3.16, Casa Software Ltd, UK). All peaks were calibrated with respect to the adventitious hydrocarbon C Is at 284.8 eV since a charge neutralizer was used to compensate for charge build-up. The charge neutralizer was set at 1.6 amps. Peak fitting was performed after background subtraction, which was carried out using an iterated Shirley-Sherwood algorithm.

### Example 1.9. Membrane Stability to H₂

To evaluate the stability of the AgTf₂N polymer electrolyte membranes in the presence of H₂, the pure gas ethylene-ethane selectivity was rapidly measured following consecutive 24-hr. periods of pure H₂ permeation. Thus, pure-gas permeation experiments of ethylene, ethane, and H₂ were performed and then repeated in that order. The permeability of each gas (and thus the ideal C₂H₄/C₂H₆ selectivity) was measured according to the method described above at a pressure of 2 bar and 35° C. Photographs were taken before and after completion of the test to determine if the membrane had developed brown discoloration, which might qualitatively indicate the presence of Ag⁰ nanoparticles, which have been reported to form when Ag⁺ is reduced. XPS measurements were conducted as described previously on samples following completion of the H₂ stability test as well as on control samples of the same membrane casting which were not exposed to H₂ in order to assess whether a change in the average oxidation state had occurred.

### Example 1.10. Effect of UV Light on Ag⁺

To verify the sensitivity of the aforementioned XPS technique to Ag⁰, membrane samples containing Ag⁺ were exposed to 5, 10, 15, 20, and 30 min of UV light using the same UV crosslinking oven and settings utilized in the membrane synthesis procedure. Images were taken of each sample to record changes in the membrane color. XPS was performed on a control sample not exposed to UV light, a sample exposed for 15 min, and a sample exposed for 30 min to assess progressive changes in the oxidation state of Ag⁺ with additional UV light exposure.

### Example 1.11 Membrane Stability to C₂H₂

A similar procedure to Example 1.9 and Example 1.11 was performed to evaluate the stability of the AgTf₂N polymer electrolyte membranes in the presence of C₂H₂. The pure gas ethylene-ethane selectivity was rapidly measured following consecutive periods of permeating a mixture of 5 vol % C₂H₂ in nitrogen at 2 bar and 35° C. from a pre-mixed cylinder through the membrane. The pure gas permeabilities and selectivity were measured at 2 bar and 35° C. as described above, and photographs were taken before and after the test.

### Example 1.12 Membrane Stability to H₂S

To evaluate the stability of the AgTf₂N polymer electrolyte membranes in the presence of H₂S, a procedure similar to Example 1.9 was performed. The pure gas ethylene-ethane selectivity was rapidly measured following consecutive 24-hr. periods of permeating a mixture of 300 ppm H₂S in nitrogen at 2 bar and 35° C. from a pre-mixed cylinder through the membrane. The pure gas permeabilities and selectivity were measured at 2 bar and 35° C. as described above, and photographs were taken before and after the test. XPS measurements were performed as described previously to evaluate whether Ag⁺ had been converted to Ag₂S following H₂S exposure.

### Example 1.13. Experimental Results

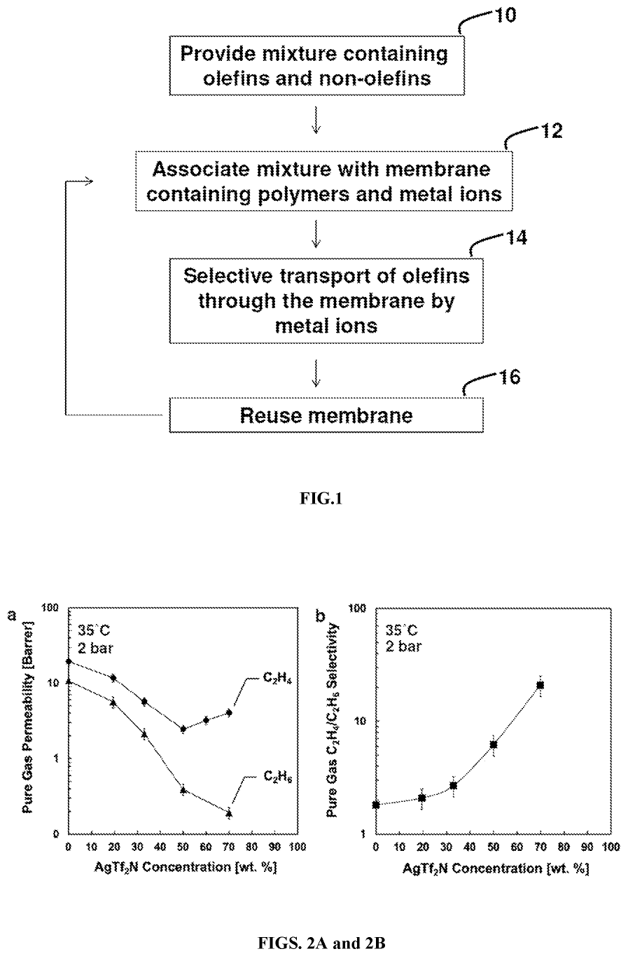

The pure gas permeabilities of ethylene and ethane in AgTf₂N-containing SPE membranes were measured over a range of AgTf₂N concentrations (FIG. 2A). Ethane permeability monotonically decreases with additional Ag⁺ content, while ethylene permeability initially decreases before rising following a minimum value at 50 wt % AgTf₂N. These phenomena are consistent with effect of salt concentration on ethylene and ethane permeability in other AgBF₄-containing SPEs. Increasing the concentration of solvated Ag⁺ in the membrane leads to a more rigid polymer matrix, with a greater fraction of ether oxygen moieties coordinated to Ag⁺ ions acting as non-permanent physical crosslinks. This crosslinking reduces the average size of free volume elements, in turn reducing the diffusivity of gases. However, the incorporation of chemically specific Ag⁺ complexing sites significantly increases ethylene solubility.

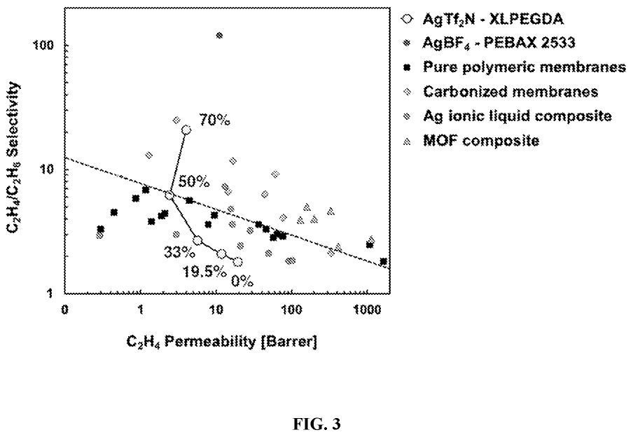

Thus, the ethylene-ethane selectivity of the membrane increases with AgTf₂N concentration (FIG. 2B). The maximum pure gas selectivity measured in the sample of highest concentration was 20.8, with an ethylene permeability of 4.0 Barrer. This separation performance places these membrane materials above the gas separation upper bound for ethylene-ethane separations (FIG. 3).

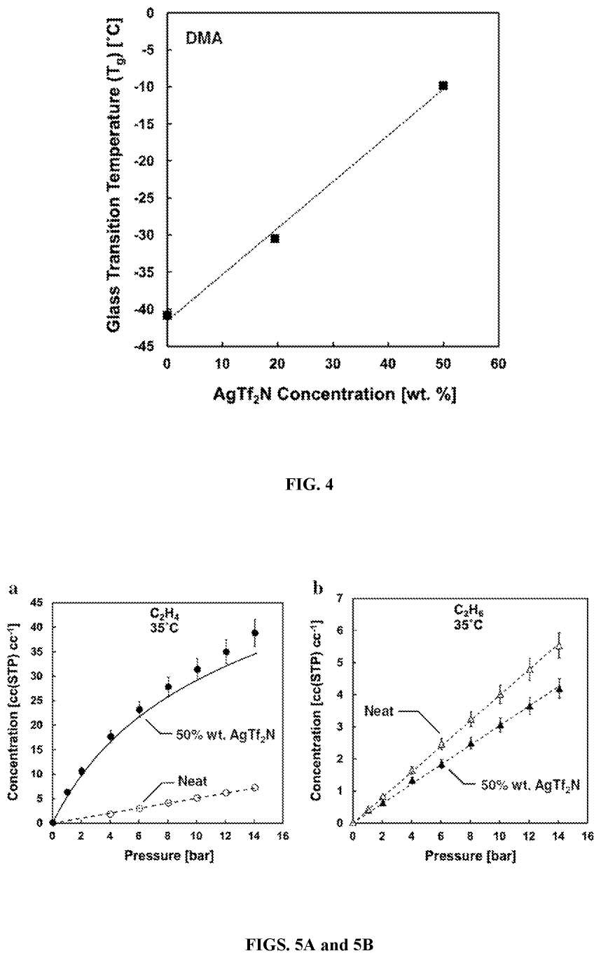

The local scale rigidity of a polymer matrix can be characterized by the glass transition temperature, with rubbery polymers above T_{g} exhibiting more long-range chain motion. The glass transition temperature of the AgTf₂N SPEs was measured for several salt concentrations (FIG. 4). T_{g} increased by 30° C. relative to the neat polymer following incorporation of 50 wt % AgTf₂N, indicating reduced chain mobility and an increasingly rigid matrix, consistent with increasing cohesive energy density. Additionally, incorporating 1-butyl-2,3-dimethylimidazolium bistriflimide ([BMMIM⁺][Tf₂N⁻]) ionic liquid was found to have a plasticizing effect, reducing T_{g} and increasing gas permeability without affecting selectivity. This Example further demonstrates a strong correlation between gas permeability and glass transition temperature of the rubbery materials.

Pure-gas measurements of the ethylene and ethane solubility were undertaken. Ethylene uptake increased substantially in comparison to the neat polymer upon the addition of 50 wt % AgTf₂N to the crosslinked polymer matrix (FIG. 5A). Ethylene solubility and the ethylene-ethane solubility selectivity increased relative to the neat polymer with the addition of 50 wt % AgTf₂N, consistent with a higher concentration of chemically specific Ag⁺ binding sites capable of binding olefins but not paraffins. Dual-mode sorption behavior was observed for ethylene in the AgTf₂N-containing membrane, as evidenced by the nonlinear relationship between the equilibrium concentration of absorbed ethylene and the applied pressure.

Rubbery materials, such as the AgTf₂N SPEs, are at equilibrium and typically show linear gas sorption with pressure at low pressures. Ethylene sorption in the neat polymer was linear. Both materials showed linear sorption for ethane (FIG. 5B), with slightly lower ethane solubility observed in the AgTf₂N SPE, consistent with increasing cohesive energy density.

The solubility of ethylene decreases from a maximum at infinite dilution down to a constant value at higher pressures (FIG. 6). A summary of the relevant gas permeation properties for both the neat polymer and 50 wt % AgTf₂N XLPEGDA80, including the diffusivity, diffusivity selectivity, and solubility selectivity, is given in Table 3.

Diffusion coefficients for both ethylene and ethane were calculated using independently measured pure gas permeabilities and solubilities according to the solution diffusion model. Ethylene and ethane diffusivities were lower in the sample containing salt, in the salt-containing polymer consistent with a higher cohesive energy density. One notable trend is the reversal of ethylene-ethane diffusivity selectivity from less than one to greater than one. In the neat polymer, ethylene has a higher diffusivity than ethane because diffusivity is inversely correlated with the size of the penetrant species, and ethylene has a smaller kinetic diameter than ethane (cf. Table 1). However, the diffusion coefficient for ethane is 2.5 times larger than that of ethylene in 50 wt % AgTf₂N XLPEGDA80. The selective coordination complex between with Ag⁺ can slow ethylene diffusion.

TABLE 3
Summary of ethylene and ethane transport parameters in neat and AgTf₂N-containing XLPEGDA matrices.
Permeability and solubility data were measured at 2 bar and at 35° C., and diffusion coefficients were
calculated as the ratio of permeability divided by solubility. These solubility and diffusivity values agree
well with values obtained via analysis of time lag data from the pure gas permeation experiments.

| Membrane | $\frac{{P}_{{C}_{2} {H}_{4}}}{{P}_{{C}_{2} {H}_{6}}}$ | S_{C}_{₂}_{H}_{₄} [cm³(STP) cm⁻³ bar⁻¹] | S_{C}_{₂}_{H}_{₆} [cm³(STP) cm⁻³ bar⁻¹] | $\frac{{S}_{{C}_{2} {H}_{4}}}{{S}_{{C}_{2} {H}_{6}}}$ | D_{C}_{₂}_{H}_{₄} × 10⁹ [cm² s⁻¹] | D_{C}_{₂}_{H}_{₆} × 10⁹ [cm² s⁻¹] | $\frac{{D}_{{C}_{2} {H}_{4}}}{{D}_{{C}_{2} {H}_{6}}}$ |
|---|---|---|---|---|---|---|---|
| Neat | 1.8 ± 0.2 | 0.50 ± 0.04 | 0.41 ± 0.03 | 1.2 ± 0.1 | 300 ± 40 | 200 ± 30 | 1.5 ± 0.3 |
| XLPEGDA100 | | | | | | | |
| 50% wt. AgTf₂N | 6.3 ± 0.2 | 5.2 ± 0.4 | 0.31 ± 0.02 | 17 ± 2 | 3.4 ± 0.6 | 9 ± 1 | 0.4 ± 0.1 |
| XLPEGDA80 | | | | | | | |

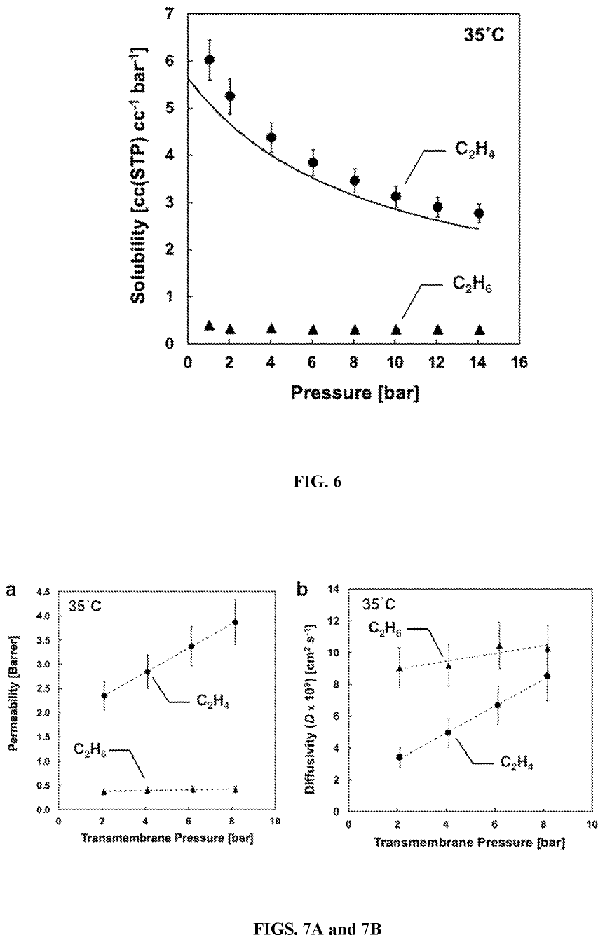

Though ethylene solubility in 50% AgTf₂N XLPEGDA80 decreased with pressure (cf. FIG. 6), pure gas ethylene permeability increased substantially with transmembrane pressure, nearly doubling after the applied pressure increased from 2 to 8 bar (FIG. 7A). Thus, the diffusivity of ethylene also increased with transmembrane pressure (FIG. 7B), indicating plasticization of the polymer matrix by ethylene. Increasing concentration of sorbed ethylene makes the polymer matrix more rubbery, leading to higher gas permeability. Ethane permeability and diffusivity in the AgTf₂N-containing polymer electrolyte remained relatively unchanged with transmembrane pressure, which could likewise be attributed to its low solubility in the polymer electrolyte.

Plasticization behavior was also observed in mixed-gas permeation conditions. Ethane permeability increased when co-permeated with ethylene relative to the pure gas experiment, and the mixed-gas selectivity was lower than that measured under pure-gas conditions for a feed composition of 65/35 vol. % ethylene/ethane, shown in Table 4. Mixed-gas ethylene permeability was lower than the pure gas case because the partial pressure of ethylene in the mixed-gas experiment was lower than the pure gas experiment for the same overall transmembrane pressure, and ethylene permeability increases strongly with ethylene partial pressure (cf. FIG. 7A).

TABLE 4
Summary of mixed-gas ethylene and ethane transport parameters for a 50 wt % AgTf₂N XLPEGDA80
sample along with corresponding pure gas values. All experiments were conducted at 35° C.

| | Mixed Gas | | | Pure Gas | | |
|---|---|---|---|---|---|---|
| Pressure [bar] | P_{C2H4} [Barrer] | P_{C2H6} [Barrer] | $\frac{{P}_{C 2 H 4}}{{P}_{C 2 H 6}}$ | P_{C2H4} [Barrer] | P_{C2H6} [Barrer] | $\frac{{P}_{C 2 H 4}}{{P}_{C 2 H 6}}$ |
| 3.5 | 2.55 | 0.71 | 3.6 | 3 ± 0.4 | 0.5 ± 0.1 | 6 ± 1 |
| 5 | 2.97 | 0.78 | 3.8 | 3.6 ± 0.4 | 0.5 ± 0.1 | 7 ± 1 |

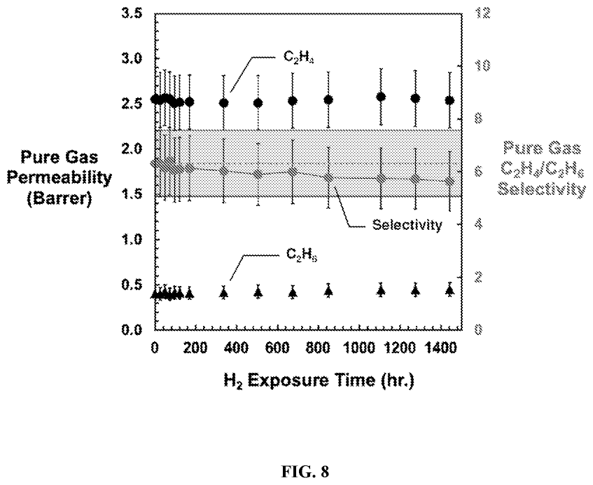

Facilitated transport membranes containing 50 wt % AgTf₂N XLPEGDA80 show remarkable resistance to chemical degradation by hydrogen gas, which has been reported to reduce Ag⁺ ions to inactive Ag⁰ (FIG. 8). After over 1400 hours of pure hydrogen permeation at 4 bar, which is the strongest reducing condition we are aware of in the open literature for silver-containing polymeric membranes, the ethylene-ethane selectivity decreased approximately 10% but was still within the uncertainty of the initial value measured prior to H₂ exposure. For comparison, hydrogen stability of the membrane was also measured with pure gas H₂ permeation at 2 bar.

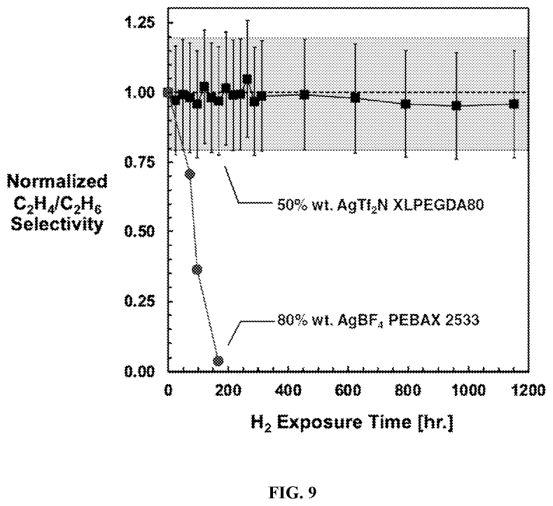

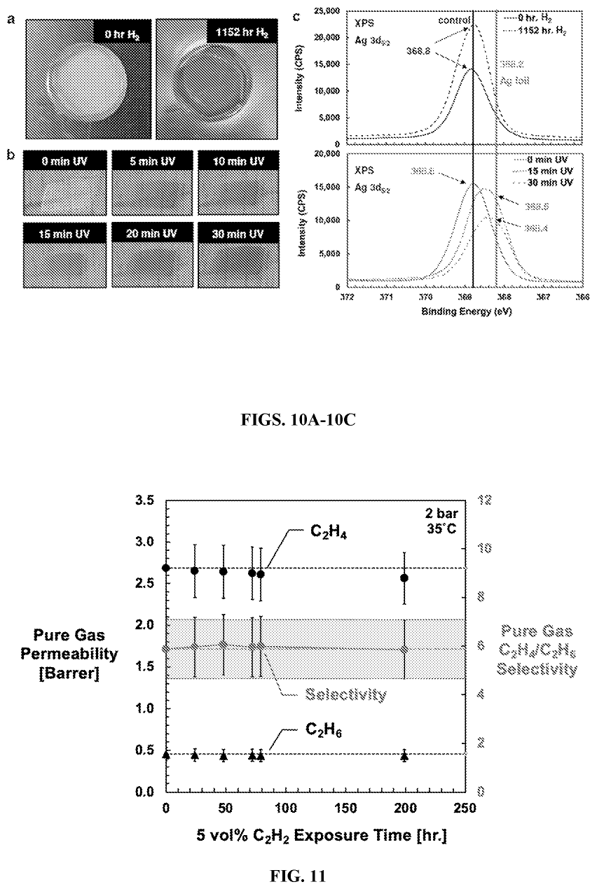

The AgTf₂N-containing membranes show a fundamental difference from the AgBF₄-containing membranes (FIG. 9). A membrane composed of 80 wt % AgBF₄ in PEBAX 2533 loses essentially all selectivity within four days of H₂ exposure at 2 bar and 35° C., while the 50 wt % AgTf₂N XLPEGDA80 membrane essentially retained its original selectivity for over seven weeks. Upon removal of the membrane sample from the cell, a color change to light reddish-brown was noted (FIG. 10A).

To determine whether this color change was the result of the formation of Ag⁰ nanoparticles, XPS measurements were conducted to evaluate the oxidation state of Ag⁺ following H₂ permeation. If Ag⁺ ions had reduced to Ag⁰, then the average oxidation state of the silver would decrease. First, a control experiment was conducted to verify that XPS is sensitive to Ag⁰ in the polymer electrolyte matrix. Samples of a 50 wt % AgTf₂N XLPEGDA80 membrane were exposed to UV light, a strong reducing agent, for up to 30 minutes (substantially longer than the 90 s exposure used during membrane synthesis.)

As seen in FIG. 10B, a significant color change was observed following UV exposure, and the membrane became more opaque. XPS spectra of the Ag 3d_{5/2} orbital confirm that the average binding energy decreased, moving closer to the binding energy of silver metal foil, consistent with decreasing average oxidation state of Ag⁺ (FIG. 10C).

Therefore, XPS can detect the formation of Ag⁰ nanoparticles in the AgTf₂N SPE matrix. Returning to the question of Ag⁺ reduction by H₂, when comparing the XPS spectra of the sample exposed to H₂ at the conclusion of the 2 bar H₂ stability test with a control sample which had been stored in a vacuum desiccator for the duration of the experiment, the binding energy remained unchanged, indicating that the silver binding energy had not been measurably changed by H₂ exposure. Thus, the observed color change does not appear to be a result of large-scale reduction of Ag⁺ to Ag⁰.

Without being bound by theory, it is hypothesized that the H₂ stability is a result of ionic aggregates of Tf₂N⁻ and Ag⁺, leading to solvation shell structures which sterically hinder the formation of Ag⁰ nanoparticles. At higher concentrations, ions are closer together. A geometric calculation, assuming that Ag⁺ ions are of uniform diameter and evenly distributed throughout the membrane, gives a distance of 9.3 Å between ions for a 50 wt. % AgTf₂N XLPEGDA80 membrane (Z. H. Liu, Y. Li, and K. W. Kowk, Polymer, 2001, 42, 2701-2706). This value is on the order of the size of Ag⁺ cations, c.a. 1.2 Å, and Tf₂N⁻ anions, c.a. 5.1-6.6 Å, which must be physically near to each other in the matrix to maintain charge neutrality. (S. Perkin, L. Crowhurst, H. Niedermeyer, T. Welton, A. M. Smith and N. N. Gosvami, Chem. Commun., 2011, 47, 6572-6574; H.-W. Cheng, P. Stock, B. Moeremans, T. Baimpos, X. Banquy, F. U. Renner and M. Valtiner, Advanced Materials Interfaces, 2015, 2, 1500159; and R. D. Shannon, Acta Cryst A, 1976, 32, 751-767.)

The AgTf₂N-containing membranes also show remarkable tolerance to C₂H₂ exposure, which has been shown to convert Ag⁺ ions responsible for facilitated transport to inactive and explosive Ag₂C₂ compounds, reducing olefin-paraffin selectivity (T. C. Merkel, R. Blanc, I. Ciobanu, B. Firat, A. Suwarlim and J. Zeid, Journal of Membrane Science, 2013, 447, 177-189). As shown in FIG. 11, throughout 200 hours of permeating a 5 vol % mixture of C₂H₂ in N₂, the pure-gas ethylene and ethane permeabilities remained constant, and the selectivity was unchanged. Upon removal from the gas permeator, no visible distinction could be made between a control sample stored in a desiccator and the sample exposed to C₂H₂.

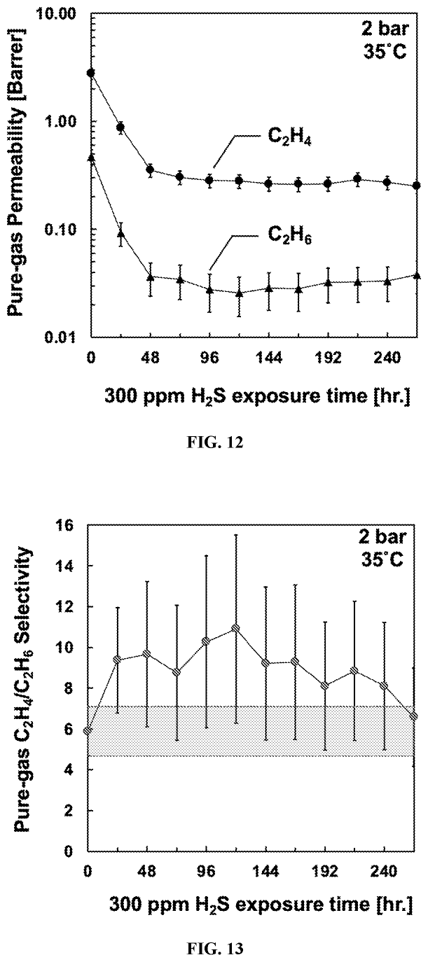

The resistance of the AgTf₂N-containing membranes to H₂S, another important contaminant, was also characterized. H₂S exposure has been previously found to reduce selectivity of Ag⁺ facilitated transport membranes by formation of crystalline and impermeable Ag₂S (T. C. Merkel, R. Blanc, I. Ciobanu, B. Firat, A. Suwarlim and J. Zeid, Journal of Membrane Science, 2013, 447, 177-189). Unlike the negligible effect of H₂ and C₂H₂ permeation on AgTf₂N-containing membrane transport properties (cf. FIG. 8 and FIG. 11), as seen in FIG. 12, permeation of a mixture of 300 ppm H₂S in N₂ for 264 hr. decreased the pure gas permeabilities of ethylene and ethane by an order of magnitude. A minimum value for the permeabilities of each gas seems to have been reached after 48 hours of H₂S permeation, after which permeability values are within the experimental error of each other.

However, as shown in FIG. 12, the selectivity of the membrane did not decrease following H₂S permeation. Due to the decreased C₂H₆ permeability in the membrane following H₂S exposure, the experimental C₂H₆ flux approached the detection limit of the gas permeator apparatus, and the experimental leak rate constituted a larger fraction of the measured C₂H₆ flux (up to approximately 20%) than initially. Therefore, the observed increase in the pure gas ethylene-ethane selectivity value following H₂S exposure is uncertain, reflected by the error bars obtained through propagation of error from the pure gas permeability values. Regardless, despite this experimental challenge, it is clear that the ethylene-ethane selectivity was not decreased following exposure to H₂S.

Following permeation of the H₂S mixture, it was observed that the AgTf₂N-containing membrane had been discolored during the experiment, changing from a transparent and clear color to an opaque, green-black color with a visible luster under the light as shown in FIG. 14A and FIG. 14B. Further, it was observed that the top and the bottom faces of the membrane were slightly different colors, with the top more green as seen in FIG. 14B and the bottom more brown as seen in FIG. 14C.

Thus, XPS measurements of both the top and bottom of the exposed sample were performed and compared with a control sample in order to assess the chemical composition of the surfaces. The oxidation state of Ag⁺, evaluated by the binding energy of the Ag3d_{5/2} peak did not measurably change following H₂S permeation because Ag is still in the +1 oxidation state in Ag₂S. However, the oxidation state of sulfur should change as the new Ag₂S species accumulates, distinct from that observed for AgTf₂N in the control sample. As shown in FIG. 14D, following permeation of 300 ppm H₂S, both the top and the bottom surface show a drastic change in binding energy for the peak corresponding to sulfur (S 2p_{3/2}), 169.0 eV for the control sample to approximately 162 eV, which is similar to a literature value of 161.7 eV for Ag₂S (L. J. Gerenser, K. E. Goppert-Berarducci, R. C. Baetzold and J. M. Pochan, The Journal of Chemical Physics, 1991, 95, 4641-4649).

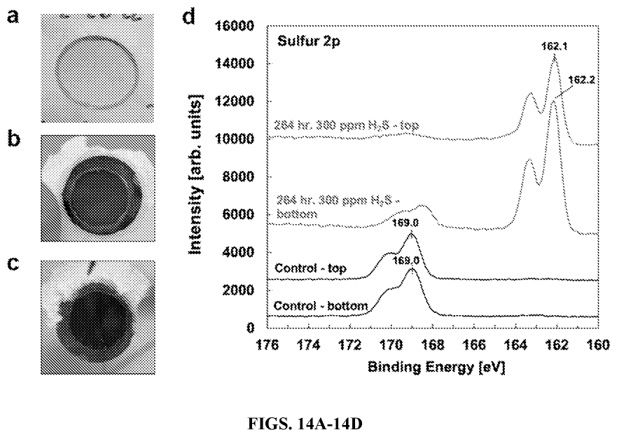

In addition, it is evident that Ag⁺ the top surface of the membrane has been more completely converted into Ag₂S because the original peak corresponding to AgTf₂N at approximately 169 eV is much more diminished than the bottom surface as shown in FIG. 14D. Thus, without being bound to theory, it is hypothesized that a crystalline layer of Ag₂S has been formed on the surface of the membrane, reducing the permeability of both ethylene and ethane through the membrane. However, this layer has not propagated throughout the entire film, so the ethylene-ethane selectivity did not decrease because the bulk of the Ag⁺ in the membrane is still in the form of AgTf₂N and active for facilitated transport (cf. FIG. 13). XPS is limited to a maximum depth of approximately 10 nm of the surface. Since a sulfur peak corresponding to AgTf₂N is still visible in the spectra of the top and bottom surfaces of the membrane exposed to H₂S, this means that there is AgTf₂N within 10 nm of the surface, and the formed Ag₂S layer is unlikely to be substantially thicker than roughly 10 nm.

In summary, preparation of dense, polymeric Ag⁺ facilitated transport membranes whose pure-gas ethylene-ethane selectivity is much less sensitive to H₂ exposure than other such membranes reported in literature has been demonstrated. Long-term H₂ stability studies were conducted with pure gas permeation of H₂ at two different pressures, with the longest experiment lasting over sixty days without a substantial decrease in ethylene-ethane selectivity. It was further found that the membranes are stable in the presence of C₂H₂. However, exposure to H₂S leads to the formation of Ag₂S on the surface and decreased permeability, though the selectivity is not decreased. The overall performance of these membranes surpasses the ethylene-ethane polymeric gas separation membrane upper bound, indicating strong performance relative to traditional gas separation membrane materials. The high ethylene-ethane permeability selectivity results primarily from increased solubility selectivity for ethylene due to facilitated transport. Ag⁺ ions act as preferential binding sites for olefins, explaining the observation of dual-mode sorption of ethylene in the AgTf₂N SPE. High olefin solubility in the AgTf₂N-containing membranes plasticizes the matrix and leads to increasing ethylene diffusivity and pure gas ethylene-ethane selectivity with pressure.

The surprising results reported herein are unexpected in the field of Ag⁺ facilitated transport membranes and indicate a potential solution to a primary barrier to commercialization of this technology. Development of H₂-stable, C₂H₂-stable, and H₂S-tolerant, facilitated transport membranes could have major industrial ramifications and could open a new chapter in the potential for Ag⁺ facilitated transport membranes to capture immediate value in mining vent streams and even compete with cryogenic distillation for olefin-paraffin separations, leading to increased process intensification and large energy savings.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

1. A method of separating olefins from a mixture comprising olefins and non-olefins, said method comprising:
associating the mixture with a membrane,
wherein the membrane comprises polymers and metal ions associated with the polymers;
wherein the metal ions mediate the transport of the olefins through the membrane by selectively and reversibly coupling with the olefins; and
wherein the olefin/non-olefin selectivity of the membrane remains within at least 80% of its original selectivity after 200 hours of exposure to a stream of hydrogen gas.

2. The method of claim 1, further comprising a step of dehydrogenating the mixture prior to associating the mixture with the membrane, wherein the dehydrogenating forms olefins from the non-olefins and generates hydrogen gas.

3-5. (canceled)

6. The method of claim 1, wherein the olefins comprise ethylene and the non-olefins comprise ethane or wherein the olefins comprise propylene and the non-olefins comprise propane.

7-10. (canceled)

11. The method of claim 1, wherein the membrane is in the form of a non-porous membrane.

12. The method of claim 11, wherein the non-porous membrane is associated with a porous support.

13-14. (canceled)

15. The method of claim 1, wherein the metal ions are selected from the group consisting of transition metal ions, silver ions, copper ions, gold ions, nickel ions, iron ions, manganese ions, zinc ions, and combinations thereof.

16-19. (canceled)

20. The method of claim 1, wherein the metal ions are in the form of metal salts and wherein the metal salts are selected from the group consisting of AgF, AgBr, AgI, Ag₂CO₃, AgHCO₃, AgNO₂, AgNO₃, Ag₂SO₄, AgClO₄, AgCN, AgSCN, AgOCN, AgAsF₆, AgSbF₆, AgPF₆, AgP(CF₃CF₂)₃F₃, AgBF₄, AgB(CN)₄, AgBF₃(CF₂CF₃), AgB(C₆F₅)₄, AgTfO (AgCF₃SO₃), AgNfO (AgC₄F₉SO₃), AgTf₂N (Ag(CF₃SO₂)₂N), Ag(CF₃)₂N, AgCF₃CO₂, AgN(CN)₂, AgN(CF₃CF₂SO₂)₂, AgFSI (Ag(FSO₂)₂N), AgC(CN)₃ and combinations thereof.

21-26. (canceled)

27. The method of claim 1, wherein the metal ions have concentrations of at least 15 wt % relative to the polymers.

28-34. (canceled)

35. The method of claim 1, wherein the polymers are selected from the group consisting of polyamides, polyimides, polyetherimide, polypyrrolones, polyesters, polyethers, poly(vinyl methyl ketone) poly(ether ether ketone), polymethylene oxides, polyethylene oxides, poly(trimethylene oxides), poly(tetramethylene oxides), poly(propylene oxides), polyethylene glycols, poly(ethylene imine), polyalkylene sulfides, sulfone-based polymers, nitrile-based polymers, polymeric organosilicones, fluorinated polymers, polydimethylsiloxane, polydiethylsiloxane, polydi-iso-propylsiloxane, polydiphenylsiloxane, polyethersulfone, polyphenylsulfone, polysulfone, polyacrylonitrile, polyvinylidene fluoride, polyphenylene oxide, polybenzimidazole, polyvinylpyrrolidone, poly(2-oxazoline), poly(ethyl methacrylate), poly(methyl methacrylate), poly(acrylic acid), polyacrylamide, poly(vinyl alcohol), poly(ε-caprolactone), poly(styrene-b-butadiene-b-styrene), chitosan, cellulose acetate, copolymers thereof, and combinations thereof.

36. The method of claim 1, wherein the polymers comprise polyethylene glycols.

37. The method of claim 1, wherein the polymers comprise molecular weights of less than about 2,000 Da when in un-cross-linked form.

38-40. (canceled)

41. The method of claim 1, wherein the polymers are in the form of a three-dimensional cross-linked network.

42. The method of claim 1, wherein the membrane provides an olefin/non-olefin selectivity of at least about 5 at 35° C. and a feed pressure of 2 bar.

43-46. (canceled)

47. The method of claim 1, wherein the stream of hydrogen gas comprises a steady stream of hydrogen gas at a feed pressure of at least 2 bar.

48. The method of claim 1, wherein the olefin/non-olefin selectivity of the membrane remains within at least 80% of its original selectivity after 100 hours of exposure to hydrogen sulfide or acetylene.

49. (canceled)

50. A membrane for separating olefins from a mixture comprising olefins and non-olefins, wherein the membrane comprises:
polymers and metal ions associated with the polymers;
wherein the metal ions mediate the transport of the olefins through the membrane by selectively and reversibly coupling with the olefins, and
wherein the olefin/non-olefin selectivity of the membrane remains within at least 80% of its original selectivity after 200 hours of exposure to a stream of hydrogen gas.

51. The membrane of claim 50, wherein the membrane is in the form of non-porous membranes.

52. The membrane of claim 51, wherein the non-porous membrane is associated with a porous support.

53-54. (canceled)

55. The membrane of claim 50, wherein the metal ions are selected from the group consisting of transition metal ions, silver ions, copper ions, gold ions, nickel ions, iron ions, manganese ions, zinc ions, and combinations thereof.

56-59. (canceled)

60. The membrane of claim 50, wherein the metal ions are in the form of metal salts and wherein the metal salts are selected from the group consisting of AgF, AgBr, AgI, Ag₂CO₃, AgHCO₃, AgNO₂, AgNO₃, Ag₂SO₄, AgClO₄, AgCN, AgSCN, AgOCN, AgAsF₆, AgSbF₆, AgPF₆, AgP(CF₃CF₂)₃F₃, AgBF₄, AgB(CN)₄, AgBF₃(CF₂CF₃), AgB(C₆F₅)₄, AgTfO (AgCF₃SO₃), AgNfO (AgC₄F₉SO₃), AgTf₂N (Ag(CF₃SO₂)₂N), Ag(CF₃)₂N, AgCF₃CO₂, AgN(CN)₂, AgN(CF₃CF₂SO₂)₂, AgFSI (Ag(FSO₂)₂N), AgC(CN)₃ and combinations thereof.

61-66. (canceled)

67. The membrane of claim 50, wherein the metal ions have concentrations of at least 15 wt % relative to the polymers.

68-74. (canceled)

75. The membrane of claim 50, wherein the polymers are selected from the group consisting of polyamides, polyimides, polyetherimide, polypyrrolones, polyesters, polyethers, poly(vinyl methyl ketone) poly(ether ether ketone), polymethylene oxides, polyethylene oxides, poly(trimethylene oxides), poly(tetramethylene oxides), poly(propylene oxides), polyethylene glycols, poly(ethylene imine), polyalkylene sulfides, sulfone-based polymers, nitrile-based polymers, polymeric organosilicones, fluorinated polymers, polydimethylsiloxane, polydiethylsiloxane, polydi-iso-propylsiloxane, polydiphenylsiloxane, polyethersulfone, polyphenylsulfone, polysulfone, polyacrylonitrile, polyvinylidene fluoride, polyphenylene oxide, polybenzimidazole, polyvinylpyrrolidone, poly(2-oxazoline), poly(ethyl methacrylate), poly(methyl methacrylate), poly(acrylic acid), polyacrylamide, poly(vinyl alcohol), poly(ε-caprolactone), poly(styrene-b-butadiene-b-styrene), chitosan, cellulose acetate, copolymers thereof, and combinations thereof.

76. The membrane of claim 50, wherein the polymers comprise molecular weights of less than about 2,000 Da when in un-cross-linked form.

77-81. (canceled)

82. The membrane of claim 50, wherein the polymers are in the form of a three-dimensional cross-linked network.

83. The membrane of claim 50, wherein the membrane provides an olefin/non-olefin selectivity of at least about 5 at 35° C. and a feed pressure of 2 bar.

84-86. (canceled)

87. The membrane of claim 50, wherein the stream of hydrogen gas comprises a steady stream of hydrogen gas at a feed pressure of at least 2 bar.

88. The membrane of claim 50, wherein the olefin/non-olefin selectivity of the membrane remains within at least 80% of its original selectivity after 100 hours of exposure to hydrogen sulfide or acetylene.

89. (canceled)
